(19) United States
(12) Patent Application Publication
(10) Pub. No.: US 2016/0045573 A1
(43) Pub. Date: Feb. 18, 2016

(54) NANOPARTICLES WITH EFFECTS ON ENDOTHELIAL FUNCTION AND MEMBRANE PERMEABILITY

(71) Applicant: Richard C.K. Yen, Yorba Linda, CA (US)

(72) Inventor: Richard C.K. Yen, Yorba Linda, CA (US)

(21) Appl. No.: 14/925,506

(22) Filed: Oct. 28, 2015

Related U.S. Application Data
(63) Continuation-in-part of application No. 13/560,727, filed on Jul. 27, 2012.
Application No. 13/560,727 is a continuation-in-part of application No. 13/604,770, filed on Sep. 6, 2012.
Application No. 13/604,770 is a continuation-in-part of application No. 13/605,765, filed on Sep. 6, 2012.
(60) Provisional application No. 61/573,630, filed on Sep. 10, 2011.
(60) Provisional application No. 61/627,623, filed on Oct. 14, 2011.
(60) Provisional application No. 61/573,630, filed on Sep. 10, 2011.
(60) Provisional application No. 61/627,623, filed on Oct. 14, 2011.

(51) Int. Cl. A61K 38/36; A61K 47/48

(52) U.S. Cl. CPC A61K 38/363; A61K 47/48892; A61K 47/48284

(57) ABSTRACT

The present invention relates to a method using fibrinogen-coated albumin spheres for treating a patient infected with a hemorrhagic virus. A suspension of protein nanoparticle containing submicron protein spheres is prepared and administered to the patient. The protein spheres are bound with fibrinogen molecules in vitro or in vivo. The fibrinogen-coated albumin spheres provide improved hemostatic function of a residual concentration of platelets of the patient resulting in decreasing mortality rate or decreasing morbidity of the patient. The fibrinogen-coated albumin spheres protect an endothelial function of an endothelial cell of a blood vessel of the patient resulting in improved permeability control across predetermined tissues in the patient, thereby counteracting an effect of the hemorrhagic virus on a wall of the blood vessel.

### CROSS-REFERENCE TO RELATED APPLICATION

The present application claims priority under 35 U.S.C. 119(e) based upon co-pending U.S. Provisional Application No. 61/123,481 filed Nov. 18, 2014 and co-pending U.S. Provisional Application No. 61/122,854 filed Oct. 29, 2015.

This application is a Continuation-In-Part under 35 U.S.C. §120 based upon co-pending U.S. patent application Ser. No. 13/560,727 filed on Jul. 27, 2012, U.S. patent application Ser. No. 13/604,770 filed on Sep. 6, 2012, and U.S. patent application Ser. No. 13/605,765 filed on Sep. 6, 2012. Additionally, this present application claims the benefit of priority of co-pending U.S. patent application Ser. No. 13/560,727 filed on Jul. 27, 2012, U.S. patent application Ser. No. 13/604,770 filed on Sep. 6, 2012, and U.S. patent application Ser. No. 13/605,765 filed on Sep. 6, 2012.

The entire disclosures of the above-identified prior applications are incorporated herein by reference.

### STATEMENT REGARDING PRIOR DISCLOSURES BY THE INVENTOR OR A JOINT INVENTOR

The following disclosures are submitted under 35 U.S.C. 102(b)(1)(A):

A “grace period disclosure” was published on Feb. 12, 2015 by European Commission. This publication was entitled “Commission Implementing Decision (Dec. 2, 2015)” with EU orphan designation number EU/3/15/1442. European Commission obtained the subject matter directly from the applicant not more than one year before the effective filing date of the claimed invention.

A “grace period disclosure” was published on Mar. 30, 2015 by European Medicines Agency. This publication was entitled “Public summary of opinion on orphan designation”. European Medicines Agency obtained the subject matter directly from the applicant not more than one year before the effective filing date of the claimed invention.

### BACKGROUND OF THE INVENTION

1. Field of the Invention

The present invention relates to the field of biological particles that are nanometer in size that can attach to cells that typically form barriers or boundaries between different tissues in the body, resulting in changes in function of the cells and the permeability across the cell membrane or across the tissue barrier, including having an effect in the blood-brain barrier. The present invention concerns treatment for hemorrhagic viral infections including the Ebolavirus infection.

2. Description of the Prior Art

It has been known since the early days of studies in biology that the body is organized into tissues and organs. Specialized cells such as the endothelial cells are lined up in the interior wall of blood vessels. For a long time the endothelium is recognized as the effective “barrier” (or “permeability control”) between the intravascular compartment and the extravascular compartment—the intravascular compartment being the inside of the blood vessels where the blood cells are retained, the extravascular compartment being all the cells and tissues outside the intravascular compartment, such as muscles and nerves. Since the smallest of blood vessels (i.e. the capillaries) are only 6 to 7 micron in diameter and the white cells are much bigger, it is recognized that the endothelium or some other mechanism must be in operation to allow white cells to move from the arterial side of the blood vessels to the venous side of the blood vessels and also from inside the blood vessels to the extravascular compartment when called for, such as during an infection occurring in the muscle cells outside the blood vessels. It is now recognized that the endothelium is not just a “barrier” but that it actually metabolizes and produces biological molecules important to the overall good health in the body. However, there are at the present time no products or methods which can target the barrier, “open and close” the barrier, either at the whole system level or at special points or locations within the body.

One such potential method to influence the permeability across tissues is the application of drugs or biological molecules specifically on or only to the cells that control the permeability. To do so, the “effecter drug or molecule” must be brought to the cells that control permeability and must have sufficient time to do its job before the effecter drug is removed from its target cell or cells. One such vehicle to target specific areas in vivo has been described by Yen (U.S. Pat. No. 5,609,936). Yen disclosed a method of making nanometer-sized particles which can have fibrinogen molecules attached to them during the manufacturing process. Alternatively the particles can be “blank particles” but they can capture the endogenous fibrinogen molecules within the body of the patient after the blank particles are administered to the patient. However, these published teachings are on forming plugs on the walls of blood vessels where they are leaky or where they have wounds, to effectively stop internal bleeding. Yen's disclosure did not involve any teaching on how to increase the permeability of the cell membrane of the endothelium or change the overall permeability of the barrier layer formed by the endothelial cells. In fact, Yen's disclosures to date are focused on plugging specific locations in the blood vessel walls where there are leaks or injuries, and not where there are no leaks or injuries. The present invention, in contrast, is not about injuries, and is concerned about changing the physiology of the barrier layer between different layers of tissues and cells.

The control of what substance can pass from the interior of the blood vessels to the areas outside those vessels are particularly important to the proper functioning of the brain. For example, after a meal, the sugar concentration in the blood can be much higher than when the person is hungry. Such fluctuations can be tolerated by the rest of the body. However, the brain has a “blood-brain barrier” that will not allow wide fluctuations of many essential nutrients, even glucose, which is the main energy source for brain metabolism. How the brain achieves such a tight control is the subject of many studies. The ability to influence the permeability across the blood-brain barrier may lead to new therapies to improve the condition of certain patients, e.g. patients who have neurodegenerative diseases.

In addition, the cells that typically form the barrier, which are endothelial cells, can become major factors of pathogenesis, such as in cardiovascular diseases. The vascular endothelium has an important role in vascular function and structure, mainly through the production of nitric oxide molecules which are short-lived molecules important in the vasodilation of blood vessels. The term “endothelial dysfunction” typically refers to the reduction in nitric oxide bioavailability, increased oxidative stress and the initiation or continuation of inflammation which can contribute to the development of atherosclerosis. Yen has disclosed previously that the infusion of fibrinogen-coated spheres can reduce the concentration of reactive oxygen species (ROS) in spleen cells even 51 days after one dose of fibrinogen-coated spheres administered intravenously. Therefore there is need for further teachings on how fibrinogen-coated spheres can correct endothelial dysfunction and/or improve permeability across the endothelial barrier between the intravascular compartment and the extravascular compartment.

Many approaches have been tried to treat the condition of hemorrhagic fever infections. They involve the attempt to produce vaccines or to transfuse sera from patients who have recovered (whose sera might have the antibodies against a certain virus), into patients who are newly infected. The hope is to achieve passive immunization. However, so far there are evidence that these approaches have worked. There remains to date no specific antidotes for these often fatal diseases.

The approaches tried include the following. The following agents have been studied for the prevention or treatment of Ebola infection. None have been found to be effective. The list includes: ribavirin, nucleoside analogue inhibitors of S-adenosylhomocysteine hydrolase, interferon beta, horse or goat-derived immunoglobulins, human-derived convalescent immune globulin preparations, recombinant human interferon alfa-2, recombinant human monoclonal antibody against the envelope glycoprotein of Ebolavirus, DNA vaccines expressing the Envelop Glycoprotein or the nucleocapsid protein gene of the Ebolavirus, activated protein C, recombinant inhibitor of factor Vila/tissue factor. (King et al 2014)

Indeed, using “antisera” from a survivor on a newly infected person can sometimes have the opposite effect. Published data show that infusion of the so-called “anti-serum” from an Ebola survivor into a newly infected patient not only did not help the newly infected person, but the antibodies apparently had a stimulating effect on the viruses infecting the newly infected patient.

At the present time, there are about 30 different kinds of hemorrhagic fever viruses. A specific antidote is good for only one species of virus. Work has to start all over again if one wants antidote for another species of virus.

Moreover, antidotes against viruses are notoriously difficult to make. For example, so far scientists have not been able to make a specific antidote (e.g. an antiviral agent) against the flu virus. At best, they can try to produce inactivated proteins from the virus to use as a “vaccine”. Such viral proteins are typically extracted from viruses grown in eggs or in cells, such as the “flu vaccine” to provoke hopefully an antibody production after injection into an unexposed person, for potential protection against the seasonal flu. Unfortunately, there is no guarantee that the flu vaccine will work for the season and it is expected definitely not to work on a new variety of flu virus the next season.

Therefore, there is a need for a better method of treatment for patients suffering from viral infections, particularly from hemorrhagic fever viral infections.

Therefore, a need exists for new and improved nanoparticles with effects on endothelial function and membrane permeability that can be used for treatment for patients suffering from viral infections, particularly from hemorrhagic fever viral infections. In this regard, the present invention substantially fulfills this need. In this respect, the nanoparticles with effects on endothelial function and membrane permeability according to the present invention substantially departs from the conventional concepts and designs of the prior art, and in doing so provide an apparatus primarily developed for the purpose of treatment for patients suffering from viral infections, particularly from hemorrhagic fever viral infections such as the Ebolavirus infection.

### BRIEF SUMMARY OF THE INVENTION

In view of the foregoing disadvantages inherent in the prior art now present in the prior art, the present invention provides an improved nanoparticles with effects on endothelial function and membrane permeability, and overcomes the above-mentioned disadvantages and drawbacks of the prior art. As such, the general purpose of the present invention, which will be described subsequently in greater detail, is to provide a new and improved nanoparticles with effects on endothelial function and membrane permeability and method which has all the advantages of the prior art mentioned heretofore and many novel features that result in a nanoparticles with effects on endothelial function and membrane permeability which is not anticipated, rendered obvious, suggested, or even implied by the prior art, either alone or in any combination thereof.

This invention discloses a new nanometer-sized protein particle, the method of production and the administration to a patient, which includes the intravenous route, the arterial route and the intraperitoneal route. The different routes of administration will lead to changes in the function of the endothelial cells and will also result in greater permeability across certain tissues in the body, including the blood-brain barrier, resulting in the improvement of the condition of the patient.

Additionally, the present invention is a method of treating patients who have been infected with any of the many hemorrhagic viruses, including the Ebolavirus, with a suspension of nanometer-sized protein particles which either have fibrinogen molecules already added to the particles, or which have the capacity to bind fibrinogen molecules supplied by the blood of the patient, resulting in improved mortality rate and less morbidity of the patient.

To attain this, the present invention essentially comprises a method of using fibrinogen-coated albumin spheres for treating a patient infected with a hemorrhagic virus. A suspension of protein nanoparticle containing submicron protein spheres is prepared and administered to the patient. The protein spheres are bound with fibrinogen molecules in vitro or in vivo. The fibrinogen-coated albumin spheres provide improved hemostatic function of a residual concentration of platelets of the patient resulting in decreasing mortality rate or decreasing morbidity of the patient. The fibrinogen-coated albumin spheres protect an endothelial function of an endothelial cell of a blood vessel of the patient resulting in improved permeability control across predetermined tissues in the patient, thereby counteracting an effect of the hemorrhagic virus on a wall of the blood vessel.

There has thus been outlined, rather broadly, the more important features of the invention in order that the detailed description thereof that follows may be better understood and in order that the present contribution to the art may be better appreciated.

It can be appreciated that the fibrinogen molecules are bound to the protein spheres in vivo with said fibrinogen molecules being supplied by blood of the patient. Alternatively, the fibrinogen molecules are bound to the protein spheres in vitro with the fibrinogen molecules being supplied by blood. Alternatively, the fibrinogen molecules are bound to the protein spheres in vitro or in vivo using a biological molecule solution containing fibrinogen molecules and insulin.

There are, of course, additional features of the invention that will be described hereinafter and which will form the subject matter of the claims attached.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an object of the present invention to provide a new and improved nanoparticles with effects on endothelial function and membrane permeability that has all of the advantages of the prior art and none of the disadvantages.

It is another object of the present invention to provide new and improved nanoparticles with effects on endothelial function and membrane permeability that may be easily and efficiently manufactured and marketed.

An even further object of the present invention is to provide a new and improved nanoparticles with effects on endothelial function and membrane permeability that has a low cost of manufacture with regard to both materials and labor, and which accordingly is then susceptible of low prices of sale to the consuming public, thereby making such nanoparticles with effects on endothelial function and membrane permeability economically available to the buying public.

Still another object of the present invention is to provide a new nanoparticles with effects on endothelial function and membrane permeability that provides in the apparatuses and methods of the prior art some of the advantages thereof, while simultaneously overcoming some of the disadvantages normally associated therewith.

Even still another object of the present invention is to provide nanoparticles with effects on endothelial function and membrane permeability for treatment of hemorrhagic viral infections including the Ebolavirus infection. This allows for the forming of co-aggregates with activated endogenous platelets at sites of injury inside the blood vessel of the patient at an onset of injury.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be made to the accompanying drawings and descriptive matter in which there are illustrated embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a whole-body scan of rats infused with Tc-99m-labeled fibrinogen-coated albumin spheres.

FIG. 2 is an electron microscope scan showing in the upper panel the FAS alone (using the first generation product); and in the lower panel the formation of co-aggregates (indicated by the red arrow) between FAS (indicated by the yellow star) and natural platelets.

### DETAILED DESCRIPTION OF THE INVENTION

While specific examples are given in the following sections and in this patent, it should be understood that these are only examples used to illustrate the principles taught in the patent. While people skilled in the art may use other related compounds or vary the concentrations of ingredients, such practices all fall within the scope and the spirit of the patent described herein.

It has been discovered that a comparable improvement in bleeding time produced by blank spheres in two different animal species do not necessarily lead to a comparable improvement in survival in these two species after exposure of the animals to lethal doses of irradiation.

It has been further discovered that the fibrinogen-coated spheres (containing a minimum amount of fibrinogen molecules which had been added during the manufacturing process) administered intravenously will attach to the endothelium in vivo in mice whereas blank spheres (where fibrinogen molecules are not added to the spheres during the manufacturing process) administered intravenously do not attach to the endothelium in vivo.

It has been further discovered that other biological molecules such as insulin can be used to coat nanometer-sized particles and that such combination-particles or multi-functional particles have the potential of attaching to specific cells in barrier tissues which are rich in insulin-receptors.

It has been further discovered that non-protein molecules which have anti-inflammatory properties, such as flavonoids and their glycosylated derivatives can bind to nanospheres. Examples of flavonoid aglycones include baicalein, quercetin, daidzein and genistein. The corresponding monoglycosides are baicalin, guercitrin, daidzin and genistin. In addition, puerarin and a polyglycoside (rutin) can also bind spontaneously to the nanospheres, probably through affinity for albumin molecules.

It has been further discovered that the binding or attachment of non-protein molecules which have anti-inflammatory properties will confer anti-inflammatory properties to the combination-nanospheres which can also attach to specific cells.

It has been further discovered that administration of combination-particles via the arterial route can have the potential of these combination-particles attaching to the cells of the barrier tissues on the arterial side of the organ where they can have an effect on the properties and physiology of the endothelium in the arteries, resulting in medically beneficial effects to the animal, such as improvements in the cardiovascular condition of the patient.

It has been further discovered that the administration of combination-particles via the arterial route has the potential of these combination-particles attaching to the cells of the barrier tissues on the arterial side of the organ where they can have an effect on the permeability of the barrier tissues, including the blood-brain barrier leading to medically beneficial effects such as a decrease in the dose of medicines needed to treat seizure and the decrease in the severity or the frequency of seizure of the patient.

It has been further discovered that administration of combination-particles via the intraperitoneal route can lead to these combination-particles attaching to the cells of the barrier tissues on the peritoneal membrane from the extravascular compartment where they can have an effect on the properties and physiology of the cells of the peritoneal membrane, resulting in medically beneficial effects to the patient, such as reducing the inflammation which was caused by pro-inflammatory cytokines.

It has been further discovered that administration of combination-particles via the intraperitoneal route can lead to these combination-particles attaching to the components of the barrier tissues on the peritoneal membrane where they can have a positive effect on the permeability of the barrier tissues.

Major Diseases which are Difficult to Treat Due to the Blood Brain Barrier

There are many pathological states that are difficult to treat due to the existence of or abnormalities in the blood brain barrier. Some examples include:

### Meningitis,

Brain abscess

### Epilepsy

Multiple sclerosis
Neuromyelitis optica
Late-stage neurological trypanosomiasis (Sleeping sickness)
Progressive multifocal leukoencephalopathy (PML)
De Vivo disease (also known as GLUT1 deficiency syndrome)
Alzheimer's disease
Cerebral edema
Prion and prion-like diseases
HIV encephalitis

### Rabies

The “tightness” or intactness of the blood brain barrier is due to the tight-junctions connecting the endothelial cells together. Transmembrane proteins such as occludin, claudins, junctional adhesion molecule (JAM), or ESAM, are involved. Each of these transmembrane proteins is anchored into the endothelial cells by another protein complex that includes zo-1 and associated proteins. Biological molecules designed to alter the function of any of these transmembrane proteins or anchor proteins, when delivered to the neighborhood of these proteins, should have a high likelihood of changing (either raising or lower) the permeability of the blood brain barrier.

The circulatory system in the body can be described as a closed system in the sense that the blood vessels form a network where, if the walls of the blood vessels are intact, the contents of the system, (e.g. the red blood cells) will flow within the system without ever getting outside the system. Starting from the heart, which is the pump, the blood will be pumped via the arterial system to all the organs. The walls of the arteries are thick and muscular and can withstand strong pressures, which are exerted by the contraction of the heart. Arteries would then branch out and become arterioles which are smaller but more numerous as they approach or reach inside an organ. When the organ is reached, e.g. the foot, the circulatory system becomes small capillaries which have walls that are essentially only one-cell thick and with internal diameter of about 6 to 7 micron, just big enough for a red cell to pass through, by squeezing through. There are lots of capillaries in any organ; as a result the entire bulk of the organ can be covered and can obtain the needed oxygen and nutrients. The capillaries are thin-walled so that oxygen can easily diffuse out from the well-oxygenated blood (from the intravascular compartment) and carbon dioxide can diffuse into the blood from the muscles of the foot (from the extravascular compartment). Then the various capillaries will become “bundled” together to form small veins, which in turn are “bundled” together into big veins. The veins contain deoxygenated blood and can sometimes be observed on the surface on the leg (on their way back to the heart) as the “ugly, blue” veins. The walls of the veins are typically thin and the pressure inside is low. The blood return on the venous side to the heart is done mainly by the contraction of the various muscles surrounding the veins. The squeeze on the veins by the muscles in various parts of the body will return the deoxygenated blood back to the heart (even against gravity as in the case of blood returning from the feet.)

The most common route of administration of most “injectible” drugs or compounds is done via the intravenous route for several reasons.
(1) The location of veins can be visualized much more easily than arteries. For example, by the application of a tourniquet in the upper arm, blood behind the site of the tourniquet will back up, which will cause a vein to “bulk up” and reveal itself at the skin surface.
(2) The venous system is low pressure; there will be less bleeding after a puncture with a needle and less time is needed for hemostasis than with an arterial puncture.
(3) Arterial punctures hurt much more than venous punctures.
(4) The soluble compound can be quickly circulated to all parts of the body, from the injection site in a vein to the heart first and then from the heart to every organ according to the flow of blood.

It should be noted that if the injected compound is a highly soluble compound, then the compound will not be restricted to the intravascular compartment for long. For example, injection of ethyl alcohol (e.g. at 50% concentration) via a vein will lead to a rapid dispersion of the injected alcohol into the extravascular compartment (e.g. the muscles) long before the “bolus” of injected alcohol inside the vein reaches the heart. For large molecules or molecules with high affinity for protein binding, such injected molecules will travel a longer distance from the injected site to the heart and then from the heart to the rest of the body. Even so, they will not be confined to the intravascular compartment, but will be released to other sites in according to the “oil-water partition coefficient” i.e. how well they dissolve in other locations or sites within the body.

Only drugs delivered in microscopic particles can stay within the intravascular compartment for prolong periods. Even so, the distribution between the arterial side vs the capillaries vs the venous side may not be an “even distribution”. Other factors, such as blood volume in the region and other rheological factors, such as friction between the blood and the wall of the vessel there can affect the presence or absence of particles in a particular location in the body.

In some occasions, it is desirable that a certain injected material be “restricted to” only the arterial side. This is particular important if the material is to be distributed mainly within an organ and not to other organs. Then methods of administration advantageous to such results must be considered. For example, in the treatment of cancers it may be advantageous sometimes to apply the chemotherapy only to the organ in which the cancer is located. Therefore, an “intervention radiologist” may be called to use a catheter to locate the special artery in the patient, and then the specific arteriole, through which the chemotherapy can be administered, especially when the cancer can be visualized to sit downstream directly from that arteriole. Even so, the heart can pump blood so fast through the organ that much of the chemotherapy drug will “miss” that cancer and get transported to the venous side without affecting the cancer. Also, the cancer may occupy such a large space within the organ that the radiologist is compelled to try to reach its entirety by releasing the chemotherapy from the site of a moderately large artery, thus allowing only part of the chemotherapy to go toward the cancer while other portions of the chemotherapy will bypass and miss the cancer.

Another situation where the arterial approach may be helpful can be found again in cancer therapy. In some cancer treatments, the doctor may choose to cut off the oxygen and nutrient supply of the cancer by blocking the blood coming to the cancer from the arterial side. In such cases (very rare) the radiologist can inject large particles to block the arterial blood flow to the cancer. An example of such large particles is macroaggregated albumin particles. These particles are hundreds of microns in diameter and are not to be confused with the nanometer-sized particles of this invention. The nanometer-sized particles of this invention are much smaller than even the internal diameter of the smallest capillaries and will not obstruct any blood vessel.

Arterial blood flow differs from venous blood flow in at least one major feature. Due to the high pressure within the arterial system, the friction between particles in the blood and the arterial wall is far higher than that within the venous system. It is therefore expected that it would be more difficult for particles to adhere to the blood vessels walls on the arterial side for the purpose of delivering drugs there, than is the case on the venous side of the circulatory system.

At the present time, there are very few methods available to deliver drugs preferentially to the arterial side or only to the venous side. There are even fewer methods available to deliver drugs to mainly the blood capillaries, or to the endothelium of the vascular system.

One type of nanometer-sized particles (to be called nanoparticles or nanospheres in this invention) useful for the delivery of drugs intravascularly has been disclosed by Yen. One application is the invention of “artificial platelets” which are fibrinogen-coated albumin microspheres or nanospheres. The spheres are designed for the purpose of forming co-aggregates in vivo wherever endogenous platelets are activated to form platelet plugs on the vessel walls (i.e. where wounds or leaky areas occur). The effectiveness of these fibrinogen-coated spheres on improving the survival of mice subjected to lethal doses of irradiation will have been published at the December 2014 annual convention of the American Society of Hematology. The authors are Sung and Yen, et al and the abstract is titled “Fibrinogen Coated Nanospheres Prevent Thrombocytopenia-related Bleeding. This finding is consistent with the previous findings when fibrinogen-coated spheres were evaluated with respect to bleeding time improvements and with respect to lower volumes of blood loss after a surgically induced external wound.

Surprisingly, in this model, “control spheres” do not seem to have any efficacy in the improvement of survival of mice exposed to lethal doses of irradiation. Importantly, these control spheres have the same size distribution as the Fibrinogen-coated spheres (with median diameter being about 400 nanometer). According to the data obtained from rabbits, control spheres of this (small) size should be able to capture fibrinogen molecules in vivo and produce some beneficial effects (such as improvements in bleeding time). But they showed no efficacy when survival is used as the end-point in mice. The data showed that for survival, it is important to have fibrinogen already attached to the spheres before administration to the patient.

Table One below provides a summary of the various results obtained from fibrinogen-coated spheres of various generations and sizes. It also has the results from the corresponding “control spheres” of similar size-distribution. The first generation of spheres is called Thrombospheres; the second generation is called Fibrinoplate; the third generation is called Fibrinoplate-S (the “S” denoting a “Suspension Formulation”).

The first generation product contains many populations of spheres, including a small population of spheres larger than 5 micron. It can cause obstruction in small capillaries when administered in large doses and is therefore used only in the rabbit thrombocytopenic model for proof-of-concept (that it can improve bleeding time and can reduce blood volume loss in severely thrombocytopenic rabbits.)

The second generation product is produced in China and contains spheres mainly around 2 micron in diameter with no spheres larger than 5 micron. The size distribution is designed to mimic the size of natural platelets which are about 2 micron in diameter. Due to the fact that spheres larger than 1 micron can easily settle to the bottom upon storage (unless constantly stirred) the second generation product is lyophilized. It requires reconstitution with a compatible fluid such as normal saline to result in a suspension of spheres, which must be administered to a patient within 2 hours of reconstitution of the lyophilized powder.

The third generation product is a ready-to-use formulation with a single distribution of spheres; the population has a median diameter of about 400 nanometer and smaller. There are no spheres larger than one micron in the population. This formulation has an excipient comprising sodium caprylate to protect the proteins against denaturation by heat. Another ingredient of the excipient is sorbitol, which is added to maintain an osmolarity compatible with blood. Sorbitol has the advantage that it does not turn brown as much as the other sugars when treated with heat. A dark brown suspension will result after heating if glucose or lactose were used in the excipient. This third generation product is treated with heat for 60 degrees Centigrade for 10 hours after the sphere suspension is filled into the bottle and sealed, a process called terminal pasteurization. After terminal pasteurization the product is slightly yellow in color and is esthetically acceptable.

TABLE 1
Differences in results obtained in Control Spheres of various generations

| Groups | | | | |
|---|---|---|---|---|
| of | Sphere | | | Fibrinogen- |
| Expts | Generation | Tests (species) | Control Spheres | Coated Spheres |
| 1 | 1 | Bleeding Time (Rabbit) | No Effect | Improved |
| 2 | 1 | Bleeding Volume (Rabbit) | No Effect | Improved |
| 3 | 2 | Bleeding Time (Human) | No Effect | Improved |
| 4 | 2 | Bleeding Condition (Human) | Not Done | Improved |
| 5 | 3 | Bleeding Time (Rabbit) | Improved | Improved |
| 6 | 3 | Bleeding Time (Rat) | Not Done | Improved |
| 7 | 3 | Bleeding Time (Mice) | Not Done | Improved |
| 8 | 3 | Survival after Total Body | Not Improved | Improved |
| | | Lethal Irradiation (Mice) | | |
| 9 | 3 | Intactness of Co-aggregates | No Improvement | Better than pure |
| | | with Fibrin against lysis by | | Fibrin clumps |
| | | Plasmin (in vitro) | | |
| 10 | 3 | Attachment to vessel wall | No Attachment | Attach to |
| | | after intravenous | | Endothelium |
| | | administration of | | forming batches |
| | | fluorescein-labeled spheres | | on the blood |
| | | (Mice) | | vessel interior |
| | | | | wall |

Additional comments and explanations will be provided in the following Experimental Sections.

### Experiment One

### Improvement in Survival after Total Body Irradiation in a Mouse Model

### Purpose

To compare the efficacy of control (blank) spheres with that of fibrinogen-coated spheres in a mouse irradiation model.

### BACKGROUND

Work done in the first and the second generation of spheres all showed that control spheres have no effect while fibrinogen-coated spheres of similar sizes have a positive effect on bleeding time. In addition, bleeding time can be correlated to other clinical conditions such as the volume of blood loss—which is less when fibrinogen-coated spheres are administered.

In order to improve user-friendliness, by eliminating the need to constitute a freeze-dried powder into suspension (as is needed in the second generation product) and to allow the step of terminal pasteurization which is not possible with a dry powder, a third generation product was produced. To make sure the spheres will not settle to the bottom but can be suspended by the Brownian movement of the fluid around the spheres, the sphere size is reduced, so that the median diameter is about 400 nanometer for one lot; and about 100 nanometers in subsequent lots. It is recognized that the “surface to mass” ratio will be greatly increased for a given weight of spheres, when the size of the individual sphere is reduced. It was not clear when that is achieved, nor is it obvious at that time that the small spheres (defined as having median diameter of less than 500 nanometer) can improve bleeding time or have other medically beneficial efficacy measures.

Therefore It is a total surprise when the same rabbit thrombocytopenic model that has been used to evaluate the first and second generation spheres shows that control spheres of the third generation work as well as fibrinogen-coated spheres in terms of reducing bleeding time. It was hypothesized at that time that the efficacy of the control spheres of the third generation is due to at least 3 factors. (1) Control spheres have the capacity of binding fibrinogen in vivo as well as in vitro. (2) The smaller third generation spheres flow intravascularly closer to the vessel wall than the first and second generation spheres. Wounds occur on the wall and not in the center of the blood vessel lumens; therefore even if all else is the same, the third generation product would be more effective, dose for dose. (3) The dose effect: a similar dose in terms of weight (e.g. 8 mg per kg weight of the test subject) is not the same in terms of particles—the smaller third generation product has far more particles per mg of product than the first and second generation products, and far more surface area for potential interaction with other material, such as activated platelets.

As evidence that at least some of the above hypotheses are correct, control spheres many months after their synthesis have been mixed with human plasma. It was shown that indeed control spheres can bind spontaneously more than one coagulation factor when mixed with anticoagulated human plasma. Therefore, control spheres merely convert themselves in vivo to fibrinogen-coated spheres using the patient's own endogenous fibrinogen (plus perhaps additional beneficial biological molecules from the blood).

Regarding the second hypothesis, there is no direct evidence at that time that smaller spheres do flow nearer to the blood vessel wall than larger spheres of the first and second generation. Only recently do we have the ability to observe the flow of particles inside the capillaries of a live animal, such as will be reported below. The chemical structure of the first and second generation control spheres are similar to that of the third generation control spheres. Therefore there is no question that they all can bind the patient's own fibrinogen in vivo. The lack of efficacy of the control spheres of the first and second generations (even when they are converted to fibrinogen-bound spheres in vivo) may indeed be explained by where they flow in the blood stream, i.e. too far away from the wall of the blood vessel to have a positive effect.

Regarding the dose effect: the amount of material administered is comparable in terms of the weight of material administered per kilogram weight of the patient. The first generation product is dosed according to the number of particles that can be counted by the Coulter Counter. It contains some large spheres (larger than 5 micron) but all the spheres greater than 0.8 micron can be counted in a standard Coulter Counter. Spheres smaller than 0.8 micron cannot be counted accurately by the technology of that time. Although spheres smaller than 0.8 micron are not counted, they are not separated from the bulk. The concentration of the (counted) spheres (i.e. larger than 0.8 micron) is adjusted to 2.5 billion (i.e. 2.5E+9) spheres per ml, which is approximately the estimated concentration of platelets in a unit of platelet to be transfused to a patient. The effective dose is 3 ml of suspension (7.5 billion spheres of larger than 0.8 micron diameter) per kg weight of the rabbit. A dose of 3 ml per kg is equivalent to 210 ml (which is about the volume of one unit of platelet) to be administered to a 70 kg man. Since the spheres smaller than 0.8 micron are not separated or removed from the population of the spheres larger than 0.8 micron, the total number of spheres administered (of all sizes) is actually larger than 7.5 billion spheres administered per kg weight of the rabbit.

The theoretical number of spheres administered can be calculated assuming a density of 1.0 gram per ml for the spheres. If the dose is a standard 8 mg of sphere per kg weight of the rabbit, and the size of the spheres are uniform, the number of particles administered will be as follows: (a) 1.22 E+8 spheres per kg (for spheres uniformly 5 micron); (b) 1.91E+9 spheres per kg (for spheres that are uniformly 2 micron); (c) 2.39E+11 spheres per kg (for spheres that are uniformly 0.4 micron in diameter); (d) 1.53E+13 spheres per kg (for spheres that are uniformly 0.1 micron in diameter.)

By contract the total surface area of all the spheres that have been administered as a dose of 8 mg spheres per kg weight of the animal will be as follows (equal to the surface area of one sphere x the number of spheres administered in a dose of 8 mg spheres/kg): (a) 96; (b) 240; (c) 1200; (d) 4800 square cm per kg wt of the animal for spheres that are of uniform diameters of (a) 5; (b) 2; (c) 0.4; (d) 0.1 micron, respectively.

It is not clear for the purpose of hemostasis, nor is it obvious at this time that the number of particle administered is the most important factor for its medicinal effect, or whether the total surface area, or if the combination of these factors would also be important contributors to the beneficial effect observed. However, the above calculations show that the first generation product, when administered as a comparable dose in terms of weight of spheres administered per kg weight of the patient, will have far fewer particles and will have far smaller total surface area provided by the administered spheres, than the second generation spheres. In turn, the second generation product will have fewer particles and smaller total surface area provided by the spheres to interact with activated platelet or other surfaces inside the body, when compared to the third generation product.

Therefore, the goal of this experiment is to evaluate if control spheres of the third generation are as effective as fibrinogen-coated spheres, in mice rendered severely thrombocytopenic by a lethal dose of total-body irradiation.

### Materials and Methods

Control spheres (CS) and Fibrinogen-Coated albumin spheres (FAS) were manufactured using the methods disclosed for the third generation spheres, with median diameter about 400 nanometers and even smaller dimensions. Mice were irradiated with a dose of total-body irradiation corresponding to LD95 for the mouse strain. The mice were divided randomly into 3 groups and administered, (a) control fluid using normal saline at 1 ml per kg weight of the mouse; (b) CS administered at a dose of 8 mg CS (i.e. 1 ml) per kg weight of the mouse; (c) FAS administered at a dose of 8 mg FAS (i.e. 1 ml) per kg weight of the mouse. The dose was administered 3 times to each mouse, on day 1, 5, 10 after the day of irradiation.

### Results

Mortality rate of the three groups was scored on day 45 after irradiation. The mortality rate for group (a), (b) and (c) was 95%, 90% and 40%, respectively. There is no statistically significant difference between the mortality rate between group (a) and (b) but the P value between (a) and (c) was less than 0.01.

### Discussion

The data obtained from rabbits and from human volunteers using the first and second generation products (with median diameter larger than one micron) showed that CS has no efficacy but FAS has efficacy. In contrast, the data obtained from using nanospheres (third generation of either CS or FAS) on rabbits show that with respect to bleeding time, CS works as well as FAS. Since bleeding time has been well correlated with other clinical end-points, it is expected that CS of the third generation should also improve survival in mice.

Therefore, the result of this experiment in mouse using the CS of the third generation is a surprise in that it has little or no efficacy with respect to survival in mice. The result is not expected and is totally non-obvious in view of the Prior Art. Whether this is a reflection on the difference between the species (rabbit vs mouse) or whether additional factors are involved will be discussed in details below. It raises the question whether CS would have efficacy in human patients with different medical conditions. It may be possible that different end-points in clinical efficacy will demand different amounts (minimal amount) of pre-attached fibrinogen on the spheres (added during the manufacturing process) before the spheres can show efficacy. It is expected that if the clinical end-point is a more challenging one, efficacy will be achieved with spheres pre-attached with a higher amount of fibrinogen, while “easier cases” (perhaps bleeding time) can show efficacy with spheres carrying a lower concentration of fibrinogen (as perhaps is the case with control spheres getting the fibrinogen from an internal sources, i.e. supplied by the endogenous supply of fibrinogen from the patient.)

It is known that patients with multiple kinds of injuries, e.g. burn plus irradiation, have higher mortality rates when compared to patients with only exposure to the same dose of irradiation. It is not surprising that perhaps CS will show efficacy in the “cleaner cases” such as surgical patients who have adequate pre-operational care but CS will not show important benefits in the “harder cases” such as patients who have multiple organ failures. Obviously different clinical trials will have to be designed to answer those questions.

### Experiment Two

### Visualization of Sphere Movement Inside a Live Mouse

### Purpose

To evaluate the hypothesis that nanospheres flow inside blood vessels nearer to the endothelium than the center of the blood vessel lumen.

### Background

The hypothesis consistent with rheological principles that large particles flow essentially in the center of the lumen of a tube while smaller particles move closer to the wall of a tube may hold true for the red blood cells (about 7 microns in diameter) and the platelets (about 2 microns in diameter). However, the CS and the FAS are much smaller particles and may be less flexible than blood cells. Therefore direct observation may be the only way to evaluate how well the hypothesis holds up in a live animal with blood vessels that can contract or dilate under various physiological demands (such as oxygen demand and oxygen tension inside the blood vessel and outside the blood vessel). In addition, blood vessels including capillaries are not straight tubes and can therefore mix up the particles quite well when they flow within these flexible and flexing vessels.

### Materials and Methods

Albumin spheres of the first and second generation are large enough to be easily visualized under a light microscope. In addition, these first and second generation products have enough auto-fluorescence (natural fluorescence from the chemical bonds within the spheres) that they can be seen clearly as green objects under a fluorescent microscope. In contrast, the nanospheres of the third generation are typically too small to be seen as individual spheres with the light microscope and additional labeling with a strong fluorescent label is necessary for them to be visualized under fluorescent conditions. For this experiment, only spheres of the third generation are used.

For this experiment, CS and FAS with median diameter of about 200 nanometer were manufactured according to the Prior Art (using a fibrinogen solution at a concentration of 1.0 mg fibrinogen per ml; and adding this fibrinogen solution in a ratio of one volume of fibrinogen solution to three volumes of blank sphere suspensions during the manufacturing steps). Both CS and FAS were then labeled with fluorescein using the FITC (fluorescein isothiocynate) method.

A specially designed microscope that can visualize particles as small as 200 nanometer is used to detect the passage of nanospheres through blood capillaries in a live mouse. The mouse is under anesthesia so that it does not move. The ear is lightly compressed under the optics of the microscope while the fluorescein-labeled CS or FAS are administered via the tail vein at 8 mg spheres/kg weight of the animal.

### Results

Due to the special condition needed to visualize the small capillaries within the ear of a live mouse, it is possible that the blood vessels may be compressed and even slightly distorted and that the flow of particles inside the blood vessels is not the same as in normal conditions.

After an expected delay of about 10 minutes from the start of the infusion of labeled spheres, a steady stream of fluorescent particles begin to show up under the microscopic view. The particles are evenly spaced and not particularly concentrated at the edge of the capillary. However, few red blood cells are seen entering and passing through the capillary under observation. This suggests that the capillary may have been compressed by the instrument. However, the blood vessel under observation is most likely a capillary, or a very small blood vessel, because its diameter is because its diameter is about 30 to 40 times larger than the diameter of the passing fluorescent particles.

Despite various manipulations, the instrument is not able to focus on a small artery or a small vein. This can be due to the thickness of even a small artery or a small vein.

Within fifteen minutes of the start of the infusion of fluorecein-labeled FAS, however, the spheres entering into the capillary begin to stick to certain areas on the endothelium of the capillary and do not easily become detached by the flow of blood (or plasma) over the area. The patches of green spheres appear to gather on the wall of the capillary. There are about 100 spheres per patch and the batch does not seem to continue to grow bigger in size. The patches are distinct with respect to spatial distribution and do not become confluent. Occasionally, new patches are formed as additional spheres come inside the capillary. The total area under observation which are occupied by patches of green (stuck spheres) is about 10 to 20% of the total observed area. Not all the spheres passing by are stuck; the majority of spheres move on to areas outside the observational field of the microscope.

The same mouse when observed again, revealed patches of green under the microscope for at least 2 days. Thereafter, all the green patches disappeared.

The experiment was repeated with CS. However, CS do not stick to the endothelium of the capillary. This suggests that in the mouse, the attachment of FAS to the wall of the blood vessels may be important to its hemostatic function resulting in improved survival using FAS (but not CS).

Additional FAS were synthesized using fibrinogen solutions of 0.25, 0.50, 0.75 and 1.0 mg of fibrinogen per ml (added to the blank sphere suspension at a ratio of one volume of fibrinogen solution per three volumes of the blank sphere suspension during manufacturing, as described in the Prior Art). The four preparations are then labeled with fluorescein.

The data showed that spheres prepared with a fibrinogen solution of at least 0.5 mg fibrinogen per ml will begin to stick to the endothelium of a capillary under the above condition. Under the condition of synthesis of spheres, the minimum amount of fibrinogen that is needed for the fibrinogen-coated spheres to attach to the endothelium is about 0.01 mg of fibrinogen per mg of sphere produced.

Therefore, it is expected and extrapolated from this experiment that to show efficacy over survival (and probably other indications) the spheres should have a coating of fibrinogen of at least 1% by weight of fibrinogen per unit weight of sphere.

### Discussion

The Prior Art discusses the likely situation in which the small nanospheres would flow in areas closest to the endothelium and therefore are in the best possible location where “trouble” can start (i.e. leaky areas or wounds occurring). The observation made in this experiment does not preclude the possibility that the small nanospheres do flow near the wall of the vessels when they are flowing in an uncompressed capillary, or in a vein or in an artery. However, the Prior Art does not anticipate the attachment of fibrinogen-coated nanospheres to the wall of the blood vessels and their continuous attachment (apparently without migration to other areas because the pattern of the patches remain constant from day one to day two). The critical requirement that at least about 1% by weight of fibrinogen per unit weight of spheres is also unanticipated.

The mechanism and the basis of attachment of FAS (but not CS) to the endothelium are not clear from this experiment. The mouse has not been subjected to any irradiation or other major physiological challenges. However, it may not take much stress to produce the observations seen in this experiment. Physiological stress on an animal has been known to affect the functionality of platelets. Along the same reasoning, physiological stress imposed on this mouse under study, such as anesthesia or even a slight loss in temperature control (temperature is less well-regulated when an animal is under anesthesia) may cause enough of stress on the endothelium that certain receptors are exposed, which allow the binding of the FAS to these cells. The flow of plasma or blood across the observed capillary is probably less than in other conditions. This experiment does not allow increasing the flow to see at what flow rate or pressure gradient would the FAS not able to attach to the wall. Also the experiment does not allow us to visualize what is going on in arteries (regardless of size) and veins (regardless of size.)

The usual receptor of binding with fibrinogen is the GPIIb/IIIa receptor. It is expected that the normal array of receptors that have influence over fibrinogen binding to be involved in the process of FAS attachment to the wall of the vessel. Since the capillary is essentially only one cell thick and the cell is the endothelium, it can be appreciated that the cell bound with the FAS is the endothelial cell. It can be appreciated that the binding of the fibrinogen-coated nanosphere will influence the physiology of the bound cell.

The data suggest that there is a correlation between the attachment of spheres to the endothelium and the improvement of the treated animal in survival after a lethal dose of irradiation. The attachment of spheres observed here is totally unexpected but it can be used as a screening method which is relatively easy and economical to perform, to evaluate what compounds are useful to improve survival, before more involving experiments are done such as direct measures of survival after exposure to lethal dose of total-body irradiation.

Comments on the Various Models and Indications where Sometimes CS Works and Sometimes not

Table 1 above contains a summary of FAS consistently working and showing efficacy whether the end-point is bleeding time, survival or volume of blood loss, or improvement in the bleeding conditions of hematological patients.

However, the efficacy is unpredictable when CS is used.

Here are some reasons why CS functionality can vary:

1. One difference between CS and FAS is that fibrinogen is attached during the manufacturing process and so the “blank spheres” to which fibrinogen is added are always spheres that are newly synthesized. In fact, if the fibrinogen solution is added within one hour of the appearance of turbidity, the dilution of the concentration of ethyl alcohol by the addition of the fibrinogen solution will redissolve many (still incompletely cross-linked) spheres. Ideally the fibrinogen is added after two hours of synthesis of the spheres, Therefore, the spheres are all about two hours “old.”

2. It has been shown that CS stored for months after the date of manufacturing can still capture fibrinogen and other coagulation factors. The CS used in the mouse in Experiment Two was manufactured about two years before the experiment. It is not clear if that sample of CS can still bind fibrinogen in vitro or in vivo. Further experiments on the effect of time and storage on the capacity of CS to bind coagulation factors needs to be done.

3. When an indication for CS can be established, the anticipated method to assess “activity of the CS” would be the in vitro binding of fibrinogen when the CS is mixed with a solution of fibrinogen, at various times after synthesis of the CS. This work has not been done yet because there is no expectation prior to Experiment Two here that CS will not show efficacy in a major medical indication such as improved survival after a lethal dose of irradiation.

4. While it is true that CS can bind more than fibrinogen molecules (i.e. other coagulation factors and other biological molecules) when mixed with plasma in vitro, it is not certain that the same degree of binding, or the same coagulation factors will bind equally well in vivo. It is also not clear that the binding of fibrinogen or other important factors occur equally well in the mouse as in the rabbit. Different patients may also have different concentrations of the various factors in vivo.

5. On the question of using bleeding time as a method to predict whether a product will show efficacy in other major indication, some indications may require more fibrinogen bound per sphere than others. Therefore, FAS has the advantage in that the minimal amount would have been bound already during the manufacturing process. When CS is administered, the amount of fibrinogen or other biological molecules that will bind in vivo to convert the CS to a FAS-like particle may be sufficient to improve bleeding time but not sufficient to improve other medical conditions.

### Experiment Three

### Binding of Insulin Molecules and Other Biologically Active Substances to Nanospheres to Influence Permeability Across Cell Membranes and Permeability Across Barrier Tissues

### Purpose

To synthesize dual-purpose spheres which can attach to barrier tissues to influence their physiology including permeability across the cell membrane and permeability control across the cell layer between two different tissues.

### Background

A number of compounds have been used to evaluate the intactness of the adult and newborn blood brain barrier. By measuring uptake in the brain, or the minimum intravenous dose needed to cause a certain physiological effect in the brain, it can be shown that certain compounds can affect the blood brain barrier. Compounds that have been used to study the blood-brain barrier include the following: acetamide, antipyrine, benzyl alcohol, butanol, caffeine, cytosine, diphenyl hydantoin, ethanol, ethylene glycol, heroin, mannitol, methanol, phenobarbital, propylene glycol, thiourea, and urea. Therefore it can be appreciated that any change in the permeability of the blood brain barrier caused by the dual-purpose spheres of this invention can be evaluated by measuring the uptake of the above compounds. Alternatively it can be appreciated that the “minimal effective dose” of other drugs can be studied, which have been shown to affect the uptake of any of the above mentioned compounds.

### Material and Methods

Recombinant human insulin expressed in yeast can be purchased from Sigma (Product 12643).

Nanospheres with attachment of more than one kind of biological molecules on the spheres are synthesized as follows: one volume of non-dialyzed human serum albumin solution (diluted with water to about 6.5%) was mixed with 0.5 volume of glutaraldehyde solution (at 0.1 mg per ml). Thereafter 1.3 volume of a desolvation solution was added resulting in a total of 2.8 volume of fluid. At this step the mixture of protein solutions is non-turbid. Thereafter 1.3 volume of the same desolvation solution was added, resulting in a total of 4.1 volume of turbid sphere suspension. One example of a desolvation solution is comprised of ethyl alcohol at 75%, glutaraldehyde at 0.5 mg per ml, and the balance is with water. After approximately two hours of incubation to achieve completion of the cross-linking of the internal bonds of the spheres, the blank spheres in the suspension will become resistant to resolubilization when the concentration of alcohol is reduced. Thereafter, a solution which is a mixture of at least two types of biological molecules was added at a volume of 1.0 volume to result in a total of 5.1 volume of a suspension containing multiple-purpose spheres. One example of the “two-biological-molecules solution” is comprised of fibrinogen at 1.0 mg per ml and also insulin at 1.0 mg per ml. Thereafter, the content of alcohol in the suspension is reduced to less than 5% by dialysis in a dialysis bag against water and then sorbitol is added to achieve an osmolarity of the suspension which will be compatible with plasma.

FITC-labeling of the fibrinogen-insulin nanospheres was performed using the standard methods.

### Results

The above multiple-purpose-nanosphere suspension was assayed for the presence of fibrinogen and insulin on the spheres by an immunoassay method. The data showed that both fibrinogen and insulin molecules are present on or in the spheres. The assay cannot distinguish where the molecules are located, whether they are on the surface or inside the spheres. In this patent the term or concept of “attachment” will be used without any preference regarding the location where each or both of the molecules are attached to or on the sphere.

The microscope used to observe the flow of sub-micron particles in the capillaries of the live mice cannot be used to directly to observe the flow of similar sub-micron particles in the artery. Therefore the intravenous administration was used first by infusing a dose of 8 mg of the multiple-purpose sphere/kg weight of the mouse via the tail vein. Within 10 minutes, one can observe the appearance of the nanospheres flowing through the capillary under observation, as described in the previous experiment. However, the pattern of the patches appears to be different from the pattern of the patches observed with fibrinogen (only) nanospheres. The data suggests that the multiple-purpose nanospheres attach to more sites in the endothelial surface than are available to nanospheres containing only fibrinogen.

The fibrinogen-insulin-nanospheres were then administered via the surgically-exposed left common carotid artery at a dose of 8 mg multiple-purpose spheres/kg weight of the animal. This artery is immediately ligated with a suture after the administration of the nanospheres, to stop bleeding. A capillary is chosen from the ear for observation under the microscope. Of significant interest is that it takes over 20 minutes before the fluorescent nanospheres are observed flowing through the one capillary under observation and attaching to the wall of that capillary. The delay in appearance is consistent with the fact that the nanospheres entering via the left common carotid artery will take a longer route because that artery feeds into the organs in the head. The nanosphere will have to travel through the corresponding venous system in the head before they will go to the heart where they are then mixed with the entire blood volume returning from all parts of the animal. The “first pass” can easily miss that particular capillary under observation.

The concentration of the nanospheres in the plasma entering the capillary under observation in the microscope appears to be significantly less than is observed when the nanospheres were administered via the tail vein. This suggests that a substantial number of nanospheres have attached to the arterial walls before the remainder of the nanospheres had a chance to mix with the animal's blood volume at the heart (and then distributed to the ear). Although the endothelium of the arteries cannot be visualized with the same microscope the data suggested that the fibrinogen-insulin nanospheres do attach to the arterial wall.

It is an intention to sacrifice the animal at various times after the infusion of the above multiple-purpose nanospheres to see if there are attachments of the nanospheres to the arteries. However, it is not sure at this time if the steps needed to fix and stain an artery may detach the nanospheres from the internal surface of the arteries.

### Comment

Whether the attachment of fibrinogen-insulin nanospheres to the arterial wall would have an effect on the permeability of the cell or have an effect on the permeability of the endothelial barrier isolating the intravascular compartment from the extravascular compartment is being evaluated currently. One will, in particular, study the effect on the blood brain barrier. The approach will be as follows: in animals prone to seizure, the dose of diazepam (Valium) needed to control or calm the seizure can be assessed accurately with the tracing from a EEG (electroencephalogram) machine. It is an intention to give a dose of fibrinogen-insulin nanospheres sufficient to attach enough nanospheres to the arterial side of the blood vessels in the brain (via infusion at the left common carotid artery). It is also an intention to induce seizure in the animal using the accepted methods for the animal model. The dose of diazepam administered will then be studied via conventional ways (i.e. intravenously) that is needed to control the seizure in the brain. If attachment of the fibrinogen-insulin nanospheres has a positive effect, it can be expected that the dose of diazepam needed to control seizure to be inversely proportional to the dose of fibrinogen-insulin nanospheres administered. The control group of animals will receive either normal saline, or control spheres.

There is also an intention to study the effect of blank spheres (CS), fibrinogen (only)-nanospheres, insulin (only)-nanospheres, and combinations of other biological molecules of which the endothelium is known to have receptors. One direct approach to evaluate the effect from any of the combination-nanospheres on the intactness of the blood brain barrier is to infuse any of the test compounds mentioned in a previous section and then assay the concentration of the same compound inside the brain, after administration of the combination-nanospheres.

### Experiment Four

### Binding of Non-Protein Molecules that have Anti-Inflammatory Properties to the Nanospheres to Influence Permeability Across Barrier Tissues in the Abdomen Including Resistance Against Pro-Inflammatory Cytokines

### Purpose

To synthesize multiple-purpose nanospheres which can attach to barrier tissues in the abdomen to influence their physiology including their permeability control and resistance against pro-inflammatory cytokines

### Background

A large number of compounds has been shown to have anti-inflammatory properties. These include the common steroids and non-steroidal anti-inflammatory compounds. However, oral administration or intravenous administration of steroids for a long duration can lead to undesirable effects such as the Cushing's syndrome. Targeted delivery of steroid and other compounds with anti-inflammatory properties will have considerable benefit to the patient. Non-protein compounds such as flavonoids have natural affinity to bind to albumin molecules. This experiment will study the binding of such compounds to nanometer-sized albumin spheres.

### Material and Methods

Flavonoid aglycones and their glycosylated derivatives can be purchased from commercial suppliers. Examples of flavonoid aglycones include baicalein, quercetin, daidzein and genistein. The corresponding monoglycosides are baicalin, guercitrin, daidzin and genistin. In addition, the capacity of puerarin and a polyglycoside (rutin) to bind spontaneously to the nanospheres was evaluated.

Nanospheres with attachment of one or more than one kind of anti-inflammatory molecules on the spheres are synthesized as follows: one volume of non-dialyzed human serum albumin solution (diluted with water from 25% to 6.5%) was mixed with 0.5 volume of glutaraldehyde solution (at 0.1 mg per ml). Thereafter 1.3 volume of a desolvation solution was added resulting in a total of 2.8 volume of fluid. At this step the mixture of protein solutions is non-turbid. Thereafter less than 1.3 volume of the same desolvation solution was added, resulting in a total of less than 4.1 volume of turbid sphere suspension.

Nanospheres with attachment of one or more than one kind of anti-inflammatory molecules as well as protein biological molecules (such as fibrinogen and/or insulin) on the spheres are synthesized as follows: one volume of non-dialyzed human serum albumin solution (diluted with water from 25% to 6.5%) was mixed with 0.5 volume of glutaraldehyde solution (at 0.1 mg per ml). Thereafter 1.3 volume of a desolvation solution was added resulting in a total of 2.8 volume of fluid. At this step the mixture of protein solutions is non-turbid. Thereafter less than 1.3 volume of the same desolvation solution was added, resulting in a total of less than 4.1 volume of turbid sphere suspension. After 2 or more hours of stabilization against re-solubilization, the suspension of nanospheres is then mixed with a solution containing one or more protein biological molecules (e.g. containing 1 mg of fibrinogen per ml; and/or 1 mg of insulin per ml of solution). These nanospheres will then have a multiple-purpose function, with the fibrinogen as the attachment molecule (or the insulin as the attachment molecule) co-present with the anti-inflammatory function conferred by the anti-inflammatory non-protein molecules.

Of particular importance to the synthesis of anti-inflammatory nanospheres is the fact that many of the anti-inflammatory compounds are not very water-soluble. It is therefore important that these relatively water-insoluble compounds be dissolved in an alcohol-containing fluid, such as the desolvation solution to be used here. One example of a desolvation solution is comprised of ethyl alcohol at 75% or a higher concentration (up to 95%), glutaraldehyde at 0.5 mg per ml, containing a suitable quantity of dissolved anti-inflammatory compound, with the balance being with water. After approximately two hours of incubation to achieve completion of the cross-linking of the internal bonds of the spheres, the anti-inflammatory nanospheres in the suspension will become resistant to resolubilization when the concentration of alcohol is reduced. Thereafter, the content of alcohol in the suspension is reduced to less than 5% by dialysis in a dialysis bag against water and then sorbitol is added to achieve an osmolarity of the suspension which will be compatible with plasma.

The maximal solubility of the various compounds in ethanol can be found in the published literature. It is an intention to use desolvation solutions containing 25%, 50% and 75% of the maximum concentration of each compound to produce anti-inflammatory nanospheres of mild, moderate and strong effects, respectively.

FITC-labeling of the above anti-inflammatory nanospheres will be performed using the standard published methods.

A list of pro-inflammatory cytokines can be found in the published literature and can be purchased from commercial sources.

### Result

It is an intention to obtain results after the administration of anti-inflammatory nanospheres by the intraperitoneal route. It is an expectation to obtain evidence that anti-inflammatory nanospheres can attach onto the peritoneal membrane of the animal. As such, the attachment site will be on the extravascular site, on the opposite side of the intravascular compartment.

It is also an intention to challenge the animals with the administration of pro-inflammatory cytokines. It is an expectation that the administration of anti-inflammatory nanospheres, by a variety of routes (i.e. intravenous, intraarterial, intramuscular, but mainly intraperitoneally) before, during and after the administration of pro-inflammatory cytokines to result in a less severe inflammatory response from the host than when anti-inflammatory nanospheres had not been administered.

### Comments

The attachment of nanospheres, whether on the intravascular side or on the extravascular side can lead to changes in the permeability of the cell membranes of the endothelial cell. This is because occupation of a receptor site on the cell by an effecter molecule has been shown to lead to internal biochemical chain reactions. The fact that the sphere is bulky and prevents the effecter molecule to become internalized does not mean that the internal chain reactions cannot be turned on. In fact the presence of more than one kind of effecter molecules attaching to their corresponding receptors on the cell all at the same time can have unanticipated synergistic effects on the physiology of the cell, all of which need to be studied.

The attachment of nanospheres can also be expected to affect the ability of the endothelium to maintain the intactness of barrier imposed by the entire endothelium. The increase or decrease of the permeability of barrier tissues can be utilized to benefit patients with a variety of severe medical conditions.

In use, it can now be understood that a suspension of nanometer-sized protein particles which either have fibrinogen molecules already added to the particles, or which have the capacity to bind fibrinogen molecules supplied by the blood of the patient can be used in treating patients who have been infected with any of the many hemorrhagic viruses, including the Ebolavirus.

There are many kinds of hemorrhagic fever viruses (HFV). According to the Center for Disease Control and Prevention, commonly called the CDC (page last reviewed: Jun. 18, 2013, last updated: Jun. 18, 2013), “Viral Hemorrhagic Fevers (VHF) are caused by viruses of five distinct families: Arenaviridae, Bunyaviridae, Filoviridae, Flaviviridae, and Paramyxoviridae.”

The same article from CDC continues to explain that “each of these families share a number of features:
1. They are all RNA viruses, and are all covered, or enveloped, in a fatty (lipid) coating.
2. Their survival is dependent on an animal or insect host, called the natural reservoir.
3. The viruses are geographically restricted to the areas where their host species live.
4. Humans are not the natural reservoir for any of these viruses. Humans are infected when they come into contact with infected hosts. However, with some viruses, after the accidental transmission from the host, humans can transmit the virus to one another.
5. Human cases or outbreaks of hemorrhagic fevers caused by these viruses occur sporadically and irregularly. The occurrence of outbreaks cannot be easily predicted.
6. With a few noteworthy exceptions, there is no cure or established drug treatment for VHFs.

In rare cases, other viral and bacterial infections can cause a hemorrhagic fever; scrub typhus is a good example.”

The CDC also has a branch called the Viral Special Pathogens Branch (VSPB). The CDC explains, “The following are diseases handled by the VSPB:
1. Alkhurma homorrhagic fever (AHF)
2. Chapare hemorrhagic fever (CHHF)
3. Crimean-Congo hemorrhagic fever (CCHF)
4. Ebola hemorrhagic fever
5. Hemorrhagic fever with renal syndrome (HFRS)
6. Hantavirus pulmonary syndrome (HPS)
7. Hendra virus disease
8. Kyasanur Forest disease (KFD)
9. Lassa fever
10. Lujo hemorrhagic fever (LUHF)
11. Lymphocytic choriomeningitis (LCM)
12. Marburg hemorrhagic fever
13. Nipah virus encephalitis
14. Omsk hemorrhagic fever (OHF)
15. Rift Valley fever (RVF)
16. Tick-borne encephalitis”

The present invention is a method of treating patients who have been infected with any one of the many hemorrhagic viruses, including the Ebolavirus, with a suspension of nanometer-sized protein particles which either have fibrinogen molecules already added to the particles, or which have the capacity to bind fibrinogen molecules supplied by the blood of the patient, resulting in improved mortality rate and less morbidity of the patient.

The route is by intravenous injection. The dose is 8 mg of spheres per kg weight of the patient. The dose is to be increased to 16 mg sphere/mg if the patient already show signs and symptoms of the late stages of infection (e.g. vomiting of blood or severe diarrhea.)

The dose is typically one dose every other day. But if necessary, as judged by the patient's physician to be possibly helpful, the regiment of administration can be one dose every day.

The method of producing a large quantity of the suspension of the said nanometer-sized particles has been described in at least one or more patent applications: patent application Ser. No. 13/560,727 for MASS PRODUCTION OF READY-TO-USE SUSPENSIONS OF FIBRINOGEN-COATED ALBUMIN SPHERES FOR THE TREATMENT OF THROMBOCYTOPENIC PATIENTS, and Published Patent Application No. 2009/0304804 for BIOLOGIC DEVICES FOR HEMOSTASIS.

Many of the prior art disclosed by Yen mentions the use of products for the treatment of Ebola virus. This invention discloses details on how it can be achieved with effect.

While this invention uses the term “Ebola”, it should be understood by persons skilled in the art that such a term, unless specified otherwise in the context of the text, can also mean in this disclosure “other hemorrhagic viral infections” or “other hemorrhagic viruses”. The reason is there are so many hemorrhagic viruses that it is too wordy to mention all the other viruses every time; Ebola being the most dangerous and the most notorious virus at the present time.

### Description of the Condition

### Details of the Condition

### Definition

The condition of “Ebola infection” is the state of a patient who has been exposed to the Ebola virus and who shows signs or symptoms consistent with an infection from the Ebola virus. The condition needs to be treated before laboratory confirmation of actual infection because of the rapid progression of the disease.

### Etiology

The Ebola virus is the cause of the infection. The route of infection has not been clearly defined, probably through direct contact with an object or a human or an animal contaminated with the virus.

The genus Ebolavirus is currently classified into 5 separate species all of which are RNA viruses with a lipid envelop. All are considered zoonosis and all damage the microvasculature. The virus attacks cells directly but also causes host responses which are damaging to the host. Mainly, the additive results of the infection will result in the leakage of blood vessels (King et al, 2014) leading to extensive internal bleeding and external bleeding, hence the name “hemorrhagic viruses.”

Leakage of blood from the intravascular compartment into the extravascular (internal bleeding) not only reduces the blood volume and the cellular content in the blood, it causes deposit of break-down products in the muscles and the nerves, such as hemoglobin molecules which are toxic when the hemoglobin molecules are exposed outside the red blood cells.

The virus appears to attack a large variety of tissues and cells, often leading to uncontrolled viremia, multi-organ failure, and death. (Hutchinson et al, 2007)

### Specific Characteristics; Pathophysiological, Histopathological, Clinical Characters

There are two types of exposures: (a) primary, where the patient is from or has traveled to an Ebola-endemic area; (b) secondary, where the patient has direct contact with another person who has been infected with the Ebola virus or who consumes infected bush meat.

The Ebola virus has a filamentous form with a uniform diameter of about 80 nanometers and a variable length. The filament is often folded on itself. The virus invades, replicates in and finally destroys endothelial cells, leading to massive consumption and destruction of platelets and producing a condition known as disseminated intravascular coagulation (King et al, 2014).

### Classification

There is no clear classification of the disease condition of the patient.

However, according to the International Committee on Taxonomy of Viruses, Ebolavirus is classified into 5 species. Three of them (Sudan Ebolavirus, Zaire Ebolavirus, Ivory Coast Ebolavirus) cause most of the reported deaths (ranging from 65% to 89% mortality). A fourth species (Reston Ebolavirus) has not been documented to cause human disease. A fifth species (Bundibugyo Ebolavirus) is known to cause a mortality rate of 25% in infected patients. (King et al, 2014)

### Diagnosis and Symptoms

The key to diagnosis is the history of the patient, who has recent contact with Ebola-infected patients or who comes from an Ebola-infected area. However, the following diagnostic studies are also helpful:
a) Complete blood count with differential, bilirubin, liver enzymes, blood urea nitrogen, creatinine, pH.
b) Isolating the virus. This can only be done in one of a few laboratories in the world.
c) Reverse-transcription polymerase chain reaction assay
d) Serological testing: enzyme-linked immunosorbent assay for the Ebolavirus antigens or for Immunoglobulin M and immunoglobulin G antibodies

Early signs and symptoms include: fever, pharyngitis, maculopapular rash, bilateral conjunctival injection and unstable vital signs. Later findings include: bleeding from intravenous-catheter insertion sites and mucous membranes, myocarditis and pulmonary edema. In terminal patients the signs include hypotension, anuria, tachypnea, and coma. (King et al, 2014; CDC, 2014b)

### Proposed Indication

The indication is “Treatment of Hemorrhagic Viral Infection”.

The administration intravenously of the product is expected to provide improved hemostatic function of the residual concentration of platelets so that the patient can have more time and resource to recover from the infection.

### Medical Plausibility

### Active Substance: Description of the Medicinal Product, Pharmacological Class and Mode of Action

### Description of the Medicinal Product

Fibrinogen-Coated Albumin Spheres (FAS) is a suspension of nanometer-sized protein spheres intended for use in vivo to improve the hemostasic capacity of the thrombocytopenic patient. It works by enhancing the formation of platelet plugs at wound sites or on the walls of the leaky vascular sites inside the vascular system where the patient's own residual platelets are actively being converted into activated platelets to form platelet plugs. (Yen, 2011)

The spheres by themselves are inert in vitro and in vivo. They become trapped only in sites where platelets are being activated. In vitro studies have shown that co-aggregates between FAS and natural platelets are only formed under conditions of platelet activation. In the absence of platelet-activating signals, FAS is inert. (Sung et al, 2014)

The manufacturing process is as follows: nanometer-sized albumin spheres are first formed from clinical grades of human serum albumin solutions. The spheres are then coated with human fibrinogen molecules extracted from sero-negative human plasma. Then excipients are added to achieve compatibility with the osmolarity of blood. Due to the small size of the spheres, they are suspended in the suspension by Brownian movement of the fluid and are stable for at least one year of storage in room temperature. (Yen, 2011)

After administration into the patient by the intravenous route, the bolus of spheres will further redistribute. However, because they are particles in nature, they remain confined within the intravascular compartment and do not easily leave the intravascular system. In addition, due to their small sizes (compared to platelets which are typically 2 micron in diameter) they are pushed by rheological principals toward the interior surface of the wall of the blood vessels. Since wounds or leaky areas occur at the wall and not at the center of the lumen of blood vessels, these spheres are closer to any potential “trouble spot” than perhaps even the endogenous platelets. As they redistribute within the intravascular compartment, all the internal surfaces of the vascular system would soon have circulating spheres rolling along, which will form co-aggregates whenever and wherever wounds occur or defects are formed within the endothelial lining of the blood vessels.

It is expected that some spheres will be circulated to the liver for degradation. But compared to soluble drugs, the mass of spheres subjected to liver degradation will only be that volume that actually gets carried by the blood into the liver. Pharmacokinetics studies of the spheres show that they have a very different pattern of distribution compared to conventional soluble drugs. Soluble drugs do not stay within the vascular system, far less staying close to the endothelium and moving along the endothelium. Conventional sampling of blood for pharmacokinetic studies will draw samples from the middle or the center of a large vein. Such an approach will miss the majority of the spheres which are circulating near the walls of the blood vessels, including the walls of the capillaries. Therefore, by conventional methods of blood sampling, the spheres appear to have disappeared within one day (i.e. in blood drawn from the center of large veins), but by imaging, the rest of the body still contains a majority of the spheres and the medicinal benefit is seen even 5 days after one dose of the FAS. (Yen, 2011, table three, Bleeding Time in mice on various days.)

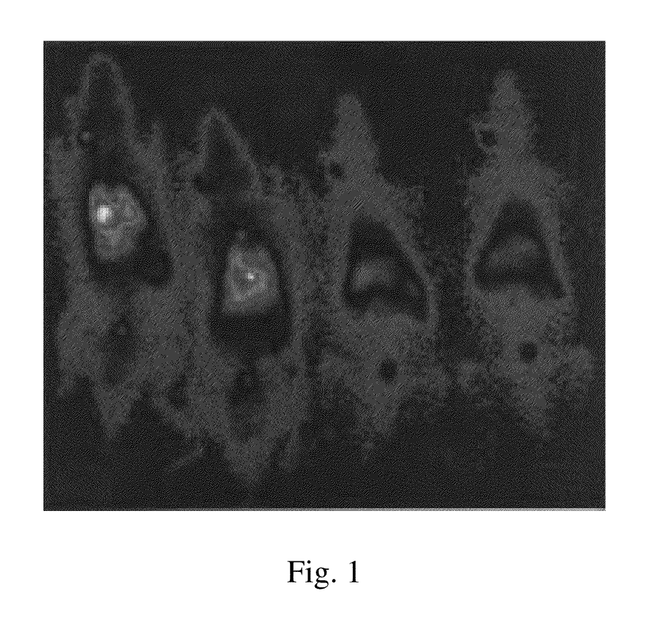

FIG. 1 is a whole body scan of rats administered with technicium-labeled FAS. Again the experiment was done with the first generation of FAS which contains a small fraction of large spheres (larger than 5 micron). Catheters were placed surgically into the internal jugular veins of 4 rats, about 15, 30, 45 and 60 minutes prior to the infusion of the radioactive FAS. Therefore rat 1 has the freshest wound compared to the others. Then the rats were administered the technicium-labeled FAS at the same time. The specific activity of the spheres used for rat 1, 2, 3 and 4 is 230, 230, 54, and 54 uCi per billion spheres, respectively.

FIG. 1 shows the distribution of radioactivity 10 minutes after infusion of the spheres. The data showed an enhanced spot of radioactivity (dark blue) over the wound in all the rats (lined up from left to right, rat 1, 2, 3 and 4, respectively.) However, rat 1 (the one on the left) has the highest concentration of radioactivity in the neck, compared to rat 2, 3, and 4, suggesting an on-going process of activated platelets capturing the newly introduced radiolabeled FAS in rat 1 more so than the other rats.

Of particular interest is that all four rats show a silhouette against the background, even though at this time the highest concentration of radioactivity is in the liver The data suggest that as early as 10 minutes after intravenous administration of the FAS, the product has been distributed to all the blood vessels over the entire body.

### Pharmacological Class:

Not applicable

### Mode of Action

Fibrinogen-Coated Albumin Spheres are inert particles in vitro. They do not form aggregates with any cells including non-activated platelets. Mixing FAS with blood does not cause changes in the chemistry of the blood or the concentration of any of the blood cells (after taking into account the effect of dilution).

Just as the fibrinogen in a healthy person's blood does not form random and un-provoked fibrin clots inside the body, the fibrinogen associated with FAS also does not form random and un-provoked fibrin clots inside the body.

In fact, the conversion of fibrinogen to fibrin is not spontaneous but it requires the action of thrombin. However, thrombin is present essentially only at a wound site. Indeed, the conversion of thrombin from prothrombin in vivo occurs typically only on the surface of activated platelets, or platelet clots. Had thrombin been allowed to recirculate freely and in large amounts, everybody would have died from extended clots all over the body soon after the first wound occurs after birth.

After infusion into the intravascular compartment, the nanometer-sized fibrinogen-coated albumin spheres will passively circulate near the endothelium, being pushed there by larger elements in the blood, e.g. the red blood cells, which by rheological principles will flow mostly along the center of the lumen of the blood vessels. In thrombocytopenic patients, the concentration of endogenous platelets is not sufficient to allow formation of an effective plug in the wall of the blood vessel when needed, or in a timely manner. Even so, as soon as some platelets are activated (i.e. at any wound site) some thrombin will be converted from prothrombin molecules there. The wound site itself will capture and bind the thrombin molecules, which will work on fibrinogen of all sources. This will occur even though the patient does not have sufficient concentrations of platelets to form effective plugs to stop internal bleeding in a timely manner.

In thrombocytopenic patients who have been administered FAS, there will be three sources of fibrinogen: (1) soluble fibrinogen, the body typically has about 2 mg of soluble fibrinogen per ml of plasma; (2) fibrinogen attached to the surface of activated platelets (because the key step of activation of platelet is the binding of fibrinogen to the surface of the platelet); (3) fibrinogen molecules associated with the FAS. In normal subjects who are not thrombocytopenic, the presence of (1) and (2) will be able to produce a plug on the wall fast enough to provide adequate hemostasis. In thrombocytopenic patients, however, the presence of (3) is needed for adequate hemostatic function. Thrombin bound to the wound site will work on all three sources of fibrinogen: the preformed mass of FAS will add bulk to the platelet plug when it is formed, in a timely manner to stop excessive leakage of blood from the intravascular compartment to the extravascular compartment.

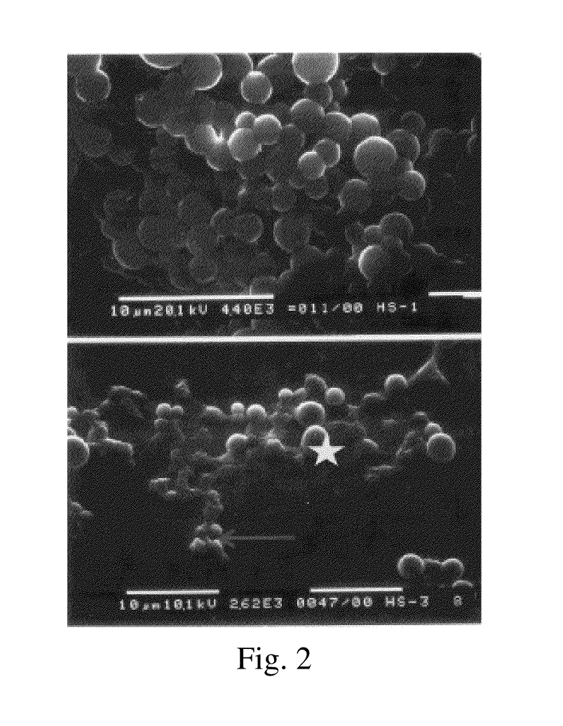

In vitro, FAS mixed with blood does not change the chemical composition of the blood, nor does it form aggregates with itself or with blood cells. However, when a mixture of FAS and platelets are activated by the addition of platelet-activating agents, such as adenosine diphosphate, co-aggregates are formed. FIG. 2 shows, in the upper panel, the scanning electron microscopy of the FAS alone (using the first generation product); and in the lower panel, the formation of co-aggregates (indicated by the red arrow) between FAS (indicated by the yellow star) and natural platelets.

Additional work done with a flow cytometer and with lethally irradiated mice at the Duke University of North Caroline showed that FAS binds to platelets in vitro, but only under conditions where platelets are activated. The authors conclude that the spheres “may prevent thrombocytopenia-related bleeding by interacting with platelets and inhibiting clot lysis. [FAS] may be helpful in many disorders including primary (immune thrombocytopenia) as well as secondary thrombocytopenia (cancer, chemotherapy, trauma, radiation injury), potentially reducing platelet transfusion, particularly in situations where platelets may not be readily available.” (Sung et al, 2014)

The work at Duke University showed that the administration of FAS can improve the survival of lethally irradiated mice, from 5% in the control group to 55% in the FAS-treated group. As a result of this work, the US National Institute of Health has signed a contract with the Duke University to expand the work. FAS is now recognized as a potential counter-measure against the terrorism using radioactive agents. The details of the work is not included in Section F and can be found in: Development of Medical Countermeasures to Enhance Platelet Regeneration and Survival Following Radiation Exposure from a Radiological/Nuclear Incident—Federal Business Opportunities: Opportunities.

### BAA-NIAID-DAIT-NIHAI2013166.

https://www.fbo.gov/index?s=opportunity&mode=form&id=3bbeba2f422433f818706e3fe0d8144d& tab=core&_cview=1
Plausibility of the Viral Infection Condition; Data with the Specific Product as Applied for in Specific Models or in Patients Affected the Condition.

It should be obvious to people skilled in the art and skilled in the practice of medicine, that the following arguments are made to show as much as possible, a superficial resemblance on the damages done by the Ebola virus and the damages done by whole body irradiation. However, there is no guarantee that the damage done by one agent is IDENTICAL to the other. It is also NOT obvious that the body reacts to the injury caused by one agent is IDENTICAL OR EVEN LIKELY TO BE IDENTICAL to the injury caused by the other agent. Therefore, even as the applicant argues in the following segment, in favor of a list of similarities, it is not obvious at all that nanosized protein spheres (with or without fibrinogen) can bring about the improved recovery, less morbidity, less mortality of the patient infected with any one or several of the hemorrhagic viruses.

Plausibility of the Orphan Condition as Compared with Another Model.

The ideal model to use for comparison with the Ebola Viral Infection would be another hemorrhagic disease where the active substance FAS has been shown to be effective. However, there are very few hemorrhagic viruses which have been studied which have effective treatments. Moreover, the active substance to be used for this Application is a novel and unique product in medicine. To date, there are no approved biological products which are non-dissolvable particles and which have the small size or the proposed indications as FAS (Fibrinogen-Coated Albumin Spheres).

Due to the high mortality rate caused by the Ebola viral infection in humans and in non-human primates, even work in vitro or the study of blood samples drawn from Ebola infected patients have to be done In BAL level 4 laboratories. Such requirements are absolutely sensible but has resulted in few detailed studies in the pathogenesis of the Ebola infection, except from laboratories with the properly trained personnel and equipment. (Sanchez, 2004)

Finding another animal or human model with which to evaluate whether FAS is likely to be effective in treating Ebola viral infection will be a challenge. The challenge is due to several reasons: (1) There are few viruses that have the rapidity of progression and lethality as the Ebola virus. (2) FAS and its predecessors have been evaluated in a number of human disease states, such as refractory idiopathic thrombocytopenia purpura, acute leukemia, acute aplastic anemia and myelodysplastic syndrome. (Li, 2006). These hematological conditions on the surface bear little resemblance to the condition of Ebola infection, except the state of severe thrombocytopenia. It is believed that the product will also benefit patients suffering from the Dengue Hemorrhagic Fever. However, the clinical plans did not have time to be finalized when the Ebola crisis has started in the EU and the USA.

However, a detailed examination of the damage done by the Ebola virus reveals that it has commonality with the damage done by high dose irradiation. In terms of the causative agents themselves, the contrast is that one is a biological agent and the other is a physical agents—one cannot be more different than the other. However, in terms of results, both can be lethal; and as such may share in pathways of damage highly detrimental in the human body.

In terms of evaluating whether a product can be effective in one disease state by comparison with its effectiveness in another disease, one may need to remember that the body has a common network of defense against insults and injuries from a large variety of causes. External causes as varied as heat, trauma, irradiation and viral infection can produce a common set of responses, such as the inflammatory response. It may not be the initial insult that causes death, but rather a combination of the initial insult and the (overactive) response by the body's own mechanisms. Therefore, a treatment such as disclosed in this invention that focuses on the initial events may stop the progression of a cascade; thereby becoming effective in diseases caused by diverse and seemingly unrelated causes. The only way to prove that such is the case, however, is by actual clinical trials in human beings, or in animal models that show strong resemblance in the pathogenesis of the infection in those animals as compared to the human infection.

In addition, internal cause of damage is often overlooked. For example, large amounts of blood extravasated from the intravascular compartment to the extravascular compartment such as large muscle groups and the nervous system may not be readily noticed by the physician and they are difficult to study. However, it is known that breakdown products of the red blood cell, including hemoglobin molecules outside the confines of the erythrocyte, can be highly toxic. (Winslow, 2013) Therefore, the initial insult can be diverse, such as from a viral infection, or it can come from irradiation. But the consequence of such diverse external insults can result in common internal injuries, such as the breakdown of the endothelial barrier and the massive consumption of platelets, or in “simple” matters as simple as electrolyte imbalance or intravascular blood volume. A medicinal product that can stop the internal insult at its early stage may be able to exert effectiveness even when the external insults appear to be very different in nature and seem unrelated.

It is believed that hemostatic control of the vascular system is a fundamental issue of health and that administration of FAS can be effective in the treatment of disease states as diverse as Ebola infection and high dose irradiation.

Comparison of the Damage Done by Ebola Infection with the Damage Done by High Dose Irradiation.

The following table 2 lists some similarities between the effect of Ebola infection and the effect of high dose irradiation.

TABLE 2

| | Effects On | Ebola Infection | High Dose Irradiation |
|---|---|---|---|
| 1 | Vascular | “Non-specific symptoms | Radiation exposure causes |
| | Permeability | rapidly develop into increased | endothelial cell changes that can |
| | | vascular permeability” | “play a critical role in mediating |
| | | (Hutchinson et al, 2007) | organ dysfunction” (Gaugler, 2005) |
| 2 | Endothelial | “The binding of the | Endothelial cells undergo |
| | cells | transmembrane glycoprotein to | autophagy after exposure to |
| | | endothelial cells may | irradiation (Kalamida et al, 2014) |
| | | contribute to the hemorrhagic | |
| | | symptoms of this disease” | |
| | | (Yang et al, 1998) | |
| 3 | Macrophages | “The initial sites of EBOV | Radiation activates NF-kappa B in |
| | | infection and replication are the | bone-marrow-derived macrophages |
| | | macrophages and DCs” | (Rithidech et al, 2012) |
| | | (Hutchinson et al, 2007) | |
| 4 | Lymphocytes | “A decrease in the number of | Irradiation induces apoptosis in |
| | | lymphocytes, which is believed | lymphocytes. (Chen et al, 2014) |
| | | to be caused by a massive | |
| | | apoptosis” (Hutchinson et al, | |
| | | 2007) | |
| 5 | Pro- | Some cytokines are elevated in | Increased by irradiation (Rithidech |
| | inflammatory | Zaire Ebola but not in Sudan | et al, 2012) |
| | cytokines | Ebola virus infection | |
| | | (Hutchinson et al, 2007) | |
| 6 | Coagulation | “Later findings may include: | “In addition to its role as a |
| | Disorder | bleeding from intravenous | selective permeability barrier, [the |
| | | puncture sites and mucous | endothelium] has many synthetic |
| | | membranes” (King et al, 2014) | and metabolic properties, including . . . |
| | | | inflammatory responses, and |
| | | | regulation of coagulation” |
| | | | (Gaugler, 2005) |
| 7 | Platelets | Severely depleted or absent in | Can be less than 10,000 per uL |
| | | Ebola patients (Sanchez et al, | (Yen, 1995) |
| | | 2004) | |
| 8 | Hemoglobin | Erythrocyte production | As low as 3 g/dL in non-survivors |
| | concentration | suppressed (Sanchez et al, | (Sung et al, 2014, FIG. 1E) |
| | | 2004) | |
| 9 | Importance | “supportive therapy with | “Supportive care in a clean |
| | of fluid | attention to intravascular | environment” (CDC, 2013) |
| | support, | volume, electrolyte, nutrition | |
| | electrolyte | and comfort care is of benefit | |
| | balance | to the patient” (King et al, | |
| | | 2014) | |
| 10 | Death | Average mortality rate (1976 to | Non-lethal doses of irradiation |
| | | 2011) is 53% and 76.6%, in | becomes lethal when animals are |
| | | Sudan and Zaire outbreaks, | weaken by administration of anti- |
| | | respectively (King et al, 2014) | platelet antibody which increases |
| | | | vascular leakage (Sung et al, 2014) |

The following discussions are more detailed discussions to put in as much favorable argument as possible between the two conditions. However it should be noted that these similarities may be superficial and that it is NOT obvious that the two conditions are identical, or that the body's reaction to the two different insults are identical, or nearly identical. Therefore, the disclosure of the present invention is not obvious based on the prior art in the scientific literature as discussed below. The following discussion merely is a discussion why it is hopeful that the nanosized protein spheres, with or without fibrinogen, should work in hemorrhagic viral infections.

### 1. VASCULAR PERMEABILITY

For a long time the endothelium of the blood vessels was recognized as merely the “inner coating” of blood vessels. In recent years, the importance of the endothelium is slowly being recognized. The endothelium is not just the regulator of vascular physiology but that of the body as a whole. As described by Gaugler, 2005: “Investigators have increasingly recognized the importance of the endothelium as a central regulator of vascular and body homeostasis.” “In addition to its role as a selective permeability barrier, it has many synthetic and metabolic properties, including modulation of vascular tone and blood flow, regulation of immune and inflammatory responses, and regulation of coagulation, fibrinolysis and thrombosis. Perturbations of endothelial structure and function result in pathological states.”

Therefore, active substances that can protect or augment the function of the endothelium should mitigate the damages done by whatever agent that inflicts damage to the endothelium, resulting in decreased morbidity or mortality of the inflicted patient.

In terms of radiation exposure, Gaugler stated, “Following radiation exposure, changes of the vasculature and more specifically of the endothelial cells were a prominent histological finding dating back to more than a century. (Gaugler, 2005)

In terms of Ebola infection, the virus is known to infect all tissues, including the endothelium. Hutchinson noted, “The initial symptoms of EBOV infection are nonspecific (i.e. fever and headache) but rapidly develop into increased vascular permeability, hypotension, coagulation disorders, and hemorrhages that often progress to multi-organ failure and death.” (Hutchinson et al, 2007, page 196)

The active substance of this Application, namely, Fibrinogen-Coated Albumin Spheres (FAS) can form co-aggregates with activated endogenous platelets at sites of injury inside the vascular system as soon as the injury starts. Preliminary data (Sung 2014, personal communication) have shown that the rate of intravascular erythrocyte loss in the initial phase after irradiation is slower in FAS-treated animals than in control animals treated with normal saline. This observation is consistent with greater leakage from the vasculature in control animals compared to FAS-treated animals.

Also the rate of platelet recovery intravascularly after the nadir is faster in FAS-treated animals than control-treatment animals, probably due to less consumption of platelets in the FAS-treated group. (Sung et al, 2014, FIG. 1D) The Sponsor believes that such will be the case in FAS-treated Ebola-infected patients because FAS works regardless of the etiology of vascular injury.

### 2. EFFECT ON THE ENDOTHELIAL CELLS

The mechanism of Ebola infection on the endothelial cells has been studied. “The transmembrane glycoprotein [of Ebola virus] was found to interact with endothelial cells.” “A murine retroviral vector pseudotyped with the transmembrane glycoprotein preferentially infected endothelial cells.” “Binding of the transmembrane glycoprotein to endothelial cells may contribute to the hemorrhagic symptoms of this disease.” (Yang et al, 1998)

In terms of the damage done by irradiation—one of the effects of radiation exposure on endothelial cells, in addition to DNA damage, is autophagy. “The vast majority of research efforts focus on agents protecting [cells] from DNA damage. Nevertheless, biological processes residing in the cytoplasm may also play an important role. Macro-autophage (autophagy) is an important biological process responsible for the turnover and recycling of long-lived proteins and dysfunctional organelles.” “Endothelial cell damage is a major effect of radiotherapy. Increased permeability leads to oedema and inflammation of organs which contributes to acute side effects.” (Kalamida et al, 2014)

Again, the Ebola viral infection will lead to destruction of endothelial cells. “The binding of the transmembrane glycoprotein to endothelial cells may contribute to the hemorrhagic symptoms of this disease.” (Yang et al, 1998)

Although the active substance Fibrinogen-Coated Albumin Spheres (FAS) is not expected by itself to be protective of the endothelial cells, the articles quoted above show that there is similarity between the effect of irradiation and the effect of Ebola virus infection on the endothelium. Therefore the efficacy of the active substance in the improvement of survival in irradiated animals may be applicable to the improvement in the survival of Ebola-infected patients.

### 3. THE EFFECT ON MACROPHAGES

At least one macrophage population has been studied specifically for the effect of irradiation. The authors study the radiation-induced oxidative damage and the inflammatory responses in bone marrow cells, including the bone-marrow derived macrophages (BMDMs). Radiation is known to activate nuclear-factor kappa B, which is a regulator of pro-inflammatory cytokines (i.e. interleukin 1-beta), IL-6, and tumor necrosis factor-alpha. Therefore, the increase or decrease in the concentrations of the various cytokines in blood sample drawn from a subject may be a consequence of irradiation on at least the macrophage population, while other cells in the body may also contribute to the inflammatory response to irradiation.

The effect of Ebola infection on the macrophage population is also known. “The initial sites of EBOV infection and replication are the macrophages and DCs” (Hutchinson, 2007, page 196.)

According to Sullivan, “Host immune responses to Ebola virus and cell damage due to direct infection of monocytes and macrophages cause the release of cytokines associated with inflammation and fever. Infection of endothelial cells also induces a cytopathic effect and damage to the endothelial barrier that, together with cytokine effects, leads to the loss of vascular integrity.” (Sullivan et al, 2003)

Although there is no direct evidence that the active substance FAS is in itself protective of the macrophage population, the articles quoted above do suggest that the irradiation model has similarities with the Ebola model as far as the damage to the macrophage population is concerned from these diverse agents.

### 4. EFFECT ON LYMPHOCYTES

In the abstract, “Edaravone protects human peripheral blood lymphocytes from gamma irradiation-induced apoptosis and DNA damage” Chen specifically discussed the effect of free radicals, a product of irradiation, on lymphocytes. “Pretreatment with edaravone increased cell viability and inhibited generation of gamma-radiation-induced reactive oxygen species (ROS) in lymphocytes exposed to 3 Gy gamma-radiation.” “Importantly, we also report that edaravone reduced gamma-irradiation-induced apoptosis through downregulation of Bax, upregulation of Bc12, and consequent reduction of the Bax:Bcl-2 ratio.” (Chen, 2014)

Mao, in collaboration with Yen, has studied the residual effects of one dose of FAS in a mouse radiation model. Surprisingly, spleen cells harvested 51 days after irradiation showed a marked difference in the generation of reactive oxygen species (ROS), depending on whether the host have been treated with control fluid (phosphate-buffered saline) or FAS at one day after irradiation. The authors hypothesized that the decreased generation of ROS in FAS-treated animals may be due to the residual effects from day one, or from the presence of an residual amount of spheres in the sinusoid or near the endothelial surfaces of the spleen, resulting in a possible beneficial effect to the host. (Mao et al, 2014)

Ebola infection also affects the lymphocyte population specifically through apoptosis. “A decrease in the number of lymphocytes, which is believed to be caused by a massive apoptosis.” (Hutchinson et al, 2007, page 196)

While the Sponsor has no direct evidence that the active substance is in itself protective of the lymphocyte population, the work by Mao et al suggests that further studies are warranted. (Mao et al, 2014)

### 5. PRODUCTION OF PRO-INFLAMMATORY CYTOKINES

There is a large body of work showing that irradiation induces the production of pro-inflammatory cytokines. For example, Pithidech et al reported on the beneficial effects of an anti-oxidant and anti-inflammatory agent called apigenin. The authors showed that irradiation produces higher levels of pro-inflammatory cytokines in control-treated than in apigenin-treated animals. They further commented that, “Additionally, the ratio of neutrophils to lymphocytes indicated that apigenin ameliorated radiation-induced hematological toxicity.” (Pithidech et al, 2010)

The production of cytokines in Ebola-infected patients has been studied in detail by Hutchinson et al. The authors reported many differences between Sudan-Ebola-infected versus Zaire-Ebola-infected patients. They ended the article by asking, “Do the differences presented here indicate differences in pathogenesis between these 2 EBOV species? Currently, there is not enough information to answer this question.” (Hutchinson et al, 2007)

There is again no expectation that the active substance FAS can have a direct effect on the generation of cytokines, whether from one species of Ebola-infection or from irradiation. However, the data from both fields do show that the body reacts to either Ebola-infection or whole body irradiation by the production of cytokines. Therefore, efficacy in the irradiation model suggests that FAS may also be effective in the Ebola infection by offering protection in some earlier stages of the injury caused by either agent.

### 6. COAGULATION DISORDER

The effect of high dose irradiation on coagulation is well known. “Following radiation exposure, changes of the vasculature and more specifically of the endothelial cells were a prominent histological finding dating back more than a century.” “In addition to its role as a selective permeability barrier, [the vascular endothelium] has many synthetic and metabolic properties, including modulation of vascular tone and blood flow, regulation of immune and inflammatory responses, and regulation of coagulation, fibrinolysis and thrombosis.” (Gaugler, 2005)

The effect of Ebola virus infection on coagulation is profound. That is why the group of viruses, of which there may be some 30 species, is called hemorrhagic viruses. (King et al, 2014)

The Sponsor believes that protection of the endothelium may be the key to the improvement of survival even when death may be caused by as diverse agents as the Ebola virus, a biological agent on the one hand, or by whole-body irradiation, a physical agent, on the other hand.

### 7. PLATELETS

The cause of thrombocytopenia can be from decreased production, or increased consumption of platelets, or both. It is quite likely that Ebola infection causes both decreased output from the bone marrow as well as consumption along injured endothelial cells. Platelets are “dramatically reduced or absent” on microscopic slides smeared with the peripheral blood from Ebola infected patients. (Sanchez et al, 2004, FIG. 1.)

The effect on platelets can also be dramatic with whole-body irradiation. The nadir is reached around Day 14 after irradiation. (Sung, 2014)

In a rabbit model of thrombocytopenia, typically with additional insult from the administration of anti-platelet antibodies, the platelet count can be less than 1% of the normal platelet count. Yet with treatment with FAS, the bleeding time and the bleeding volume can both be reduced, even at 72 hours after one dose of FAS. (Yen, 1995) Similar results are observed in a mouse model with regards to improvements in bleeding time on Day 5 after one dose of FAS administered on Day 1 after irradiation, even when the low platelet concentrations were corrected for, in animals treated with FAS compared with animals treated with control fluid, which is saline. (Sung, 2014, FIG. 1F)

Since the presence of a sufficient concentration of platelets is vital to the hemostatic function of the blood vessels, the presence of FAS in a setting of severe thrombocytopenia should be beneficial to the patient regardless of the cause of the thrombocytopenia.

### 8. HEMOGLOBIN CONCENTRATION

One consequence of vascular leakage is decrease in intravascular hemoglobin concentration, or erythrocyte mass. Sanchez also reports that “erythrocyte product is suppressed.” (Sanchez et al, 2004)

One consequence of whole-body irradiation is the hemorrhage into the intestine. Sung reports that on necropsy, intestines of animals from the control group were found to be filled with black melanotic stool, guaiac positive. Moribound mice showed hemoglobin in the 3 to 5 g/dL range, consistent with fatal gastrointestinal hemorrhage. (Sung et al, 2014, FIG. 1E.)

### 9. IMPORTANCE OF FLUID SUPPORT, ELECTROLYTE BALANCE AND ANTIBIOTICS

Supportive therapy is obviously important to very sick patients, not limited to those infected with the Ebola virus, or exposed to high doses of irradiation.

“Supportive therapy with attention to intravascular volume, electrolyte, nutrition and comfort care is of benefit to the patient” King et al reported, with regards to the Ebola infected patient. (King et al, 2014)

With regard to patients exposed to high doses of irradiation, the CDC recommends “supportive care in a clean environment.” (CDC, 2013)

Therefore, there is a strong parallel in the treatment of both types of patients who are inflicted with a condition to which no specific antidote or remedy is available.

### 10. DEATH

Death can be due to a combination of or the additive effects of several causes. It is well known that patients exposed to trauma in addition to irradiation can die from a lower dose of irradiation. In the mouse irradiation model used by Sung, a normally non-fatal dose of irradiation can become fatal if the mouse is treated at the same time with an anti-platelet antibody. This experimental approach suggest strongly that uncontrolled intravascular leakage of blood into the extravascular compartment can be a major cause of mortality. (Sung et al, 2014). Therefore, control of one function among several dysfunctions should lead to improved rates of survival.

Hemorrhage is a hallmark of Ebola infection. Death rate is 53% and 76.6% in Sudan and Zaire outbreaks, respectively (from 1976 to 2011). (King et al, 2014)

### 11. CONCLUSION

Given the similar targets of destruction from the Ebola virus and from total body irradiation, and the body's reaction to such injuries, what is effective in improving survival in one model should be beneficial to the other set of patients.

Combination Therapy Using FAS Together with Specific Antidotes.

There are no specific antidotes for the treatment of Ebola infection at the present time. (King et al, 2014) Even when methods of creating such antidotes become available, such as the production of specific antibodies against the Ebola virus, different species of Ebola may need different preparations of specific antibodies. However, if it can be shown that FAS by itself is effective in improving survival, there is no reason why FAS cannot be used in combination with the best of specific antidotes.

There is also no specific antidote for the treatment of lethal doses of irradiation at the present time. There are some treatments that are helpful, but they need to be administered immediately before exposure to the irradiation, or within one day after irradiation. If the cause of high dose irradiation is a nuclear event, there may not be any intrastructure left to treat the patients within such a narrow window of opportunity.

FAS, however, has been shown to improve survival even when used as a sole agent on irradiated animals. The advantages of using FAS over donor platelet transfusion will be discussed in the last section. As a result of the work already done at Duke University, FAS is now recognized as a potential countermeasure against radiation damage, and Duke University has signed a contract with the National Institute of Allergy and Infectious Diseases of the National Institute of Health to expand the work using FAS. The total funding amount is US$6.22 million which is to be completed in three years, starting Sep. 1, 2014. Please see: Development of Medical Countermeasures to Enhance Platelet Regeneration and Survival Following Radiation Exposure from a Radiological/Nuclear Incident—Federal Business Opportunities: Opportunities; BAA-NIAID-DAIT-NIHAI2013166. https://www.fbo.gov/index?s=opportunity&mode=form&id=3bbeba2f422433f818706e3fe0d8144d& tab=core&_cview=1

### Success of FAS in the Irradiation Model.

Earlier models used for proof-of-concept involved irradiated rabbits which were also treated with anti-platelet antibodies to bring down the platelet count to less than 1% of the normal platelet concentrations. Administration of FAS (and a predecessor of Fibrinoplate-S) has shown that bleeding time can be improved in such severely thrombocytopenic animals. (Yen, 1995, Blajchman, 1996)

Work done to correlate improvement in bleeding time with improvement in survival involved a mouse irradiation model. At least two models with mice were used to study the beneficial effects of FAS (called Fibrinogen-Coated Nanospheres in the article). In the first model where mice were exposed only to the irradiation, control mice receiving normal saline had survival rates of about 40% while the treated group had survival rates of about 80%. In a second model where the mice were further weakened by a platelet-antibody injection, survival rate of the control and the FAS-treated group was 5% and about 50%, respectively. (Sung et al, 2014)

The results are surprising because few people expect that mice exposed to lethal doses of irradiation can be rescued. Conventional teachings only focus on the perils of low concentration of blood cells intravascularly and the need to maintain blood volume. There is little teaching on the toxic effect of a massive amount of blood leaked into the extravascular compartment. Therefore, treating anemia in a patient with leaky blood vessels with erythrocyte transfusions may have underestimated the danger posed by the deposit of a massive amount of blood products in the extravascular compartment. Indeed, transfusion of platelets is not necessary helpful in a situation where the body is already overburdened by the toxic effect of a massive amount of blood in the extravascular compartment. The key in improving survival, therefore, is to reduce internal bleeding as soon as possible and to control the leakage from the intravascular compartment into the extravascular compartment.

Because FAS works by improving the hemostatic capacity of the blood vessels, less blood leaked into the extravascular compartment has resulted in almost tenfold improvement in lethally irradiated mice with dysfunctional platelets. (Sung et al, 2014)

As a result of the independent work done in the Duke University, the US government has funded through the National Institute of Health to expand the work on Fibrinogen-coated Albumin Spheres for use as a countermeasure against radiation damage.

### Justification of the Life-Threatening or Debilitating Nature of the Condition

Patients infected with the Ebolavirus show no symptoms in the early phase (which typically 3-8 days in primary cases and slightly longer in secondary cases). This will cause a delay in the patient's seeking medical help. By the time medical attention is sought after, death can occur within 8 days. The infection can spread to others around the infected patient, such as health providers and family members, through direct contact of body fluids. Therefore, it is essential that treatment be started as soon as possible of any persons with suspicions of having contracted the virus. (CDC, 2014b)

Patients who survive the Ebola infection are typically those who receive strong supportive therapy, such as intravenous fluid infusion, adequate nutrition and comfort care. The supportive care allows them to build up enough immunodefense against the virus.

Survivors typically show neutralizing antibodies in their serum. Therefore, increasing the number of survivors will increase the pool of donors whose serum can be used as passive immunization tools to help the recovery of newly infected patients.

So far there are no specific therapies available that are effective in the treatment of Ebola hemorrhagic fever. There are no commercially available vaccines. A few remedies have been tried but their efficacy cannot be established.

### Other Methods for Diagnosis, Prevention or Treatment of the Condition

### Details of any Existing Diagnosis, Prevention or Treatment Methods

### Diagnosis

The key to diagnosis is the history of the patient, who has recent contact with Ebola-infected patients or who comes from an Ebola-infected area. However, the following diagnostic studies are also helpful:
e) Complete blood count with differential, bilirubin, liver enzymes, blood urea nitrogen, creatinine, pH.
f) Isolating the virus. This can only be done in one of a few laboratories in the world.
g) Reverse-transcription polymerase chain reaction assay
h) Serological testing: enzyme-linked immunosorbent assay for the Ebolavirus antigens or for Immunoglobulin M and immunoglobulin G antibodies

### Prevention

Currently there are no vaccines available to prevent an Ebolavirus infection. Strict barrier isolation and decontamination of all body fluids are the main methods of prevention.

### Treatment

Neutralizing antibodies against the Ebolavirus have been found in survivors. Serum provided by these survivors may theoretically offer passive immunization to other patients who are newly infected. Therefore, the larger the number of patients who survive the infection, the greater the chance that passive antiserum may become available, even in a very limited amount.

The following agents have been studied for the prevention or treatment of Ebola infection. None have been found to be effective. The list includes: ribavirin, nucleoside analogue inhibitors of S-adenosylhomocysteine hydrolase, interferon beta, horse or goat-derived immunoglobulins, human-derived convalescent immune globulin preparations, recombinant human interferon alfa-2, recombinant human monoclonal antibody against the envelope glycoprotein of Ebolavirus, DNA vaccines expressing the Envelop Glycoprotein or the nucleocapsid protein gene of the Ebolavirus, activated protein C, recombinant inhibitor of factor VIIa/tissue factor. (King et al 2014)

### Justification as to why Methods are not Satisfactory

Not applicable.

### Justification of Significant Benefit

The Ebolavirus is one of the at least 30 viruses that can cause hemorrhagic fever. The common denominator of these viruses is that they cause massive consumption and destruction of platelets, leading to internal bleeding and often violent episodes of vomiting and external bleeding. Demonstration of the benefit of the product Fibrinogen-Coated Albumin Spheres in Ebola infected patients will provide insights on how to treat patients infected with these other hemorrhagic viruses to improve their survival rate.

At the present time, many patients in the intensive care units in developed countries suffer from Disseminated Intravascular Coagulation, caused by a variety of aetiologies. Treatment with platelet transfusion is often ineffective. In fact the end result of Ebola infection is like that of a patient in a state of Disseminated Intravascular Coagulation. Demonstration of the effectiveness of FAS in Ebola patients should provide data on how to use this product in this group of very sick patients. Use of the product in replacement of or in addition to donor platelets should help to control the cost of healthcare associated with the present method of platelet-transfusion for many clinical conditions.

Thrombocytopenic patients of all aetiology should benefit from this work using FAS because they all need enhanced or augmented hemostatic function. The list of patients that can benefit will include: (a) cancer patients whose platelet production ability is inhibited by either the cancer itself or by cancer treatment; (b) patients who are exposed to high doses of irradiation, either in industrial accidents, during terrorist attacks using “dirty bombs” or during a nuclear war; (c) trauma patients; (d) surgical patients who are about to undergo surgeries with expected massive blood loss; (e) any condition where platelet transfusion is expected but the platelets are not available or the patient has already developed alloimmunization from prior platelet transfusion; (f) intensive care patients including those who are developing disseminated intravascular coagulation.

Success in using FAS to control bleeding should help in reducing the cost associated at the present time with platelet transfusion. The use of FAS should reduce the direct cost of the extraction and delivery of platelet units, the cost of blood bank personnel and the cost of litigation associated with administration of the product to the “wrong patient.” The reduction in cost of national healthcare associated with the present method of donor platelet transfusions can be enormous.

In addition, the use of FAS to control early stages of bleeding should also reduce the cost associated with the use of red blood cell transfusions and plasma transfusions, because the patient bleeds less.

The following Table 3 lists some of the benefits of using Fibrinogen-Coated Albumin Spheres over donor platelet units.

TABLE 3

| | | |
|---|---|---|
| Extraction of | Requires special equipment | Synthesized entirely under aseptic |
| platelets from blood | for the isolation of platelets | conditions in a certified facility |
| | and removal of white cells in | |
| | the units | |
| Maintenance of | Needs specialized shaker, | No need for special storage |
| platelet units | needs electricity, cannot be | equipment; suitable for use in |
| | stored in freezer either | situations of little or no |
| | | electricity, e.g. after natural |
| | | disasters |
| Shelf life | Only 5 days | Over one year in room |
| | | temperature |
| Record Keeping | Need to keep track of donor, | Can be treated like other |
| | processing of unit, | medication. Routine record |
| | transportation and identity of | keeping. Can be distributed to |
| | recipient | and kept at the point-of-use, e.g. |
| | | operating room or patient floor. |
| Potential for | Even if donors are screened, | Manufactured from clinical |
| infectious agents | there is a small chance of | grades of albumin and fibrinogen |
| transmission | transmission of infectious | |
| | agents | |
| Bacterial | Can be massive after 5 days | No bacterial contamination |
| contamination of | of storage in room | |
| units | temperature | |
| Need to match with | Often needed | No need to |
| patient's blood type | | |
| Require putting in | Platelets need to be | In emergency situations, the |
| an intravenous line | administered slowly via a | product can be bloused directly |
| to the patient | pre-inserted intravenous line | into a vein. FAS will not break |
| | to the patient | apart when administered quickly |
| | | via a syringe and needle directly |
| | | into a vein |
| Transfusion | Often observed | None observed so far |
| Reactions | | |
| Alloimmunization | Often observed | Product has no cell membranes or |
| | | cell receptors. Expected to be |
| | | functional in patients who have |
| | | developed antibodies to platelets |
| Terminal | Cannot be subjected to heat | Has been treated with heat at 60 |
| pasteurization | | degrees C for 10 hours after |
| | | product is sealed inside container |
| Laboratory results | Infused donor platelets | Small size of albumin spheres not |
| | cannot be distinguished from | detected by standard laboratory |
| | patient's own platelets. | equipment. Does not interfere |
| | Difficult to tell how well | with accurate laboratory results |
| | patient is recovering in his | on any cell counts, including |
| | own platelet production | platelet counts. Allows accurate |
| | | assessment of patient's ability to |
| | | produce own platelets |
| Treatment for | Administration needs to be | Does not require skilled nursing |
| massive casualties | individualized including | to administer. Can treat massive |
| | detailed record keeping for | number of patients in situations |
| | donors, care-providers and | with little or no infrastructure or |
| | recipients | support |

Given the low toxicity profile of FAS, for patients who have low grade fevers or other symptoms but who eventually proves not have been infected with the Ebolavirus, or any of the hemorrhagic viruses, they will serve as negative controls. If there is any adverse effect due to the administration of FAS in non-Ebola-infected patients, the data from this group will be revealing. The low toxicity profile of FAS together with the potential high benefit will make it worth the risk of giving to non-Ebola-infected patients the FAS treatment. It is essential that we do not miss treating the truly Ebola-infected patient.

It is recommend that the studies be conducted as open studies so that as many Ebola-infected patients as possible can be saved. The conventional double-blinded-controlled study may not be appropriate here because of ethical reasons. In addition, the course of viral progression in the Ebolavirus infected patients will not likely be affected by psychological factors from either the patient or the health provider. The control group will be patients who cannot get the FAS at all due to any reason, including logistical problems in delivering the product to the patient.

The recommended dose will be 8 mg of spheres (1 ml) per kg weight of the patient, to be administered via an intravenous line if possible. If the patient does not have a pre-existing intravenous line, or it is not possible to insert one safely, the FAS can be directly injected into a vein. The rate is not important because the spheres cannot be broken by the pressure exerted through a needle or a syringe. The treating health provider can decide on the rate of administration of the product.

A repeat dose of 8 mg per kg can be given every other day. Pharmacokinetics studies in mice show that the effective duration (in improving bleeding time) can be as long as 5 days, in mice exposed to lethal doses of irradiation. In a safety study, daily doses of FAS in dogs result in no adverse effects: only plasma protein concentrations are raised with this daily dose, possibly due to the presence of the infused spheres in the blood sample. FAS will react to chemical tests in ways similar to soluble albumin solutions.

### Proof-of-Concept

It is an expectation the patients to be grouped for analysis purpose into two groups. The control group will consist of Ebola-infected patients who only get the standard supportive therapy such as intravenous fluid, antibiotics (for infections) and the standard transfusion products commonly practiced in that country. Patients are encouraged to receive whatever standard supportive therapy the health provider deems appropriate for the patient.

The treatment group will be patients who have a history of exposure to the Ebola virus and have started to show the signs and symptoms consistent with an Ebola infection. These patients will receive the same supportive measures as the control group mentioned above. Any person included initially in this group who later is shown not to be infected by the Ebola virus will not be included in the final analysis of the data.

It is an expectation the treated group to be statistically significantly improved over the control group in at least one of the following parameters: (a) overall mortality rate; (b) overall and specific early morbidity conditions, including the peak temperature, the duration of fever, the number of episodes and the extent of diarrhea, loss of appetite, eye inflammation, chest pain and coughing; (c) overall and specific late morbidity conditions, including blood in the stool, the number of episodes and the extent of vomiting of blood, coughing up of blood and the presence of bleeding gums.

Proof-of-concept is achieved when one or more of the above is verified.

### Pharmcokinetics

It is an expectation spheres to circulate near the inner walls of blood vessels and that a dose of 8 mg per kg will have an effective life of about 5 days. However, due to the severe nature of Ebola-infection, we should be able to achieve greater results or faster recovery of the patient if repeated doses of 8 mg/kg is administered every other day, i.e. q2 days for as long as is necessary to support the patient.

### Pharmacodynamics

It is an expectation no pharmacodynamics difference between male and female, young and old, and people of various ethnic backgrounds. This is because it is expected that no hormonal interference on the effectiveness of this product. The pathway of activation of thrombin from prothrombin appears to be well preserved among various species, including the human species. Studies conducted in a variety of animal models suggest that in all species, active thrombin molecules are attached to activated platelet clots and restricted in distribution, in vivo. Therefore, it is believed that the pharmacodynamics of this product will be the same in all species, including the human species, and it explains the safety profile of this product.

FAS is not thrombogenic: it does not cause the production of enzymes in vivo which may increase the chance of random clot formation. Repeated tests using the Wessler model, in which the internal jugular vein of an animal is ligated for a standard duration of time, to reveal any chance of FAS being thrombogenic have consistently shown that the product is non-thrombogenic. This is consistent with the fact that by themselves, the spheres are inert, in vitro and in vivo. The spheres merely work by being captured in a clot formed by activated platelets and provide the needed mass when the host has a thrombocytopenic condition.

### Clinical Efficacy

It is expected for FAS to show clinical efficacy. See proof-of-concept section

### Dose-Response Studies and Main Clinical Studies

No clinical studies have been conducted using the third generation product. Also, dose response using a variety of doses (e.g. 16 mg per kg vs 8 mg per kg) or a variety of regiments (to be given on different days after the exposure to the thrombocytopenia-inducing agent) have not been studied. The Contract signed between the National Institute of Health and the Duke University using the third generation product will aim at finding the best combination of the optimal dose and when it should be given. The contract started on Sep. 1, 2014. Work will be completed in 2017.

### Planned Clinical Studies

At the present time, there are very few Ebola-infected patients in Europe. It is hoped to enroll as many patients as possible, after signing informed consent, or in emergency situation, after obtaining legal permission as each country would allow.

It is also expected that many others will not be able to receive our product. To the extent possible, the history and development of disease stages will be used in these patients as comparison to the patients who receive FAS.

Please see the “proof-of-concept” section for further discussion.

### Clinical Safety

The second generation product has spheres that are overall larger than the third generation. The second generation product also has been lyophilized and has not been subject to a step of terminal pasteurization. Even so, there are no adverse events observed when the second generation product is administered to bleeding cancer patients and hematology patients. The third generation product is expected produce a clinical profile even safer than that of the second generation product because of the small size of the spheres in the third generation product.

### Adverse Events

It is expected that some Ebola-patients will have received this product too late to help the patient. However, if there are adverse events that happen because of the property of the third generation product, It is expected that the health provider who administered the product to report back to us and to the proper authorities.

### Serious Adverse Events and Deaths

None observed.

### Clinical Trials Using the Present Invention

The inventor is at the present time arranging with a variety of governmental agencies to plan and conduct human clinical trials as planned above. However, the incidence of Ebola viral infection or any of the other hemorrhagic fever infections are very low in the developed countries. Therefore the human clinical trials will likely be conducted in a third world, or a developing country.

It is expected that the clinical trials plans to work well in (a) improving the mortality rate; (b) decrease the morbidity in some if not all of the signs and symptoms associated with the viral infection; (c) improved health long term after recovery from the infection, as compared to untreated but recovered patients.

It can now be appreciated by the present invention that the particles can affect endothelial function and protects it suggests that the particles can protect against Ebola infection due to its destructions of the endothelium. The particles of the present invention can protect against Ebola infection because Ebola viruses work by destroying the endothelium. However, the effect of the particles of the present invention in protecting the endothelial function would counteract the effect of Ebola on the walls of the blood vessels. This is based on causation that the particles of the present invention protect inside walls of blood vessel as its function=blood vessel walls are protected=Ebola attacks the blood vessel walls=invention cures or counters the effects of Ebola.

While embodiments of the nanoparticles with effects on endothelial function and membrane permeability have been described in detail, it should be apparent that modifications and variations thereto are possible, all of which fall within the true spirit and scope of the invention. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention. And although treatment of hemorrhagic viral infections including the Ebolavirus infection have been described, it should be appreciated that the nanoparticles with effects on endothelial function and membrane permeability herein described is also suitable for treatment against all hemorrhagic viral infections.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

### REFERENCES

1) Blajchman, 1996, Evaluation of the in vivo Hemostatic Function of Human Platelets and Platelet Substitutes in a Thrombocytopenic Rabbit Model, In “Frozen Platelets and Platelet Substitutes in Transfusion Medicine” Mar. 7, 1996.
2) CDC, 2013, Acute Radiation Syndrome Fact Sheet for Physicians, http://www.bt.cdc.gov/radiation/arsphysicianfactsheet.asp, Page last reviewed: Oct. 22, 2013, Page last updated: Aug. 21, 2014.
3) CDC, 2014, Questions and Answers on Ebola, CDC: Page last reviewed: Oct. 24, 2014, Page last updated: Oct. 24, 2014.
4) CDC, 2014, Signs and Symptoms of Ebola, CDC: Page last reviewed: Oct. 18, 2014, Page last updated: Oct. 18, 2014.
5) Chen, 2014, Edaravone Protects Human Peripheral Blood Lymphocytes from Gamma Irradiation-induced Apoptosis and DNA Damage, Cell Stress Chaperones, 2014 Sep. 3.
6) Gaugler, 2005, A Unifying System: Does the Vascular Endothelium Have a Role to Play in Multi-organ Failure Following Radiation Exposure?, BJR Suppl. 2005; 27:100-5.
7) Higgins, 2014, Ebola Facts: How Many Ebola Cases are Outside of West Africa?, By ANDREW HIGGINS Oct. 17, 2014, New York Times.
8) Hutchinson, 2007, Cytokine and Chemokine Expression in Humans Infected with Sudan Ebola Virus Reprints or correspondence: Dr. Karen L. Hutchinson, Special Pathogens Branch, MS G-14, Centers for Disease Control and Prevention, 1600 Clifton Rd. NE, Atlanta, Ga. 30333 (kbh6@cdc.gov).
9) Kalamida, 2014, Important Role of Autophagy in Endothelial Cell Response to Ionizing Radiation, PLoS ONE 9(7): e102408. doi:10.1371/journal.pone.0102408.
10) Kelland, 2014, More Cases of Ebola in Europe ‘Unavoidable’, WHO says, Reuters.com, KATE'S FEED EMEA Health and Science Correspondent, Oct. 8, 2014.
11) King, 2014, Ebola Virus Infection, http://emedicine.medscape.com/article/216288-overview.
12) Li, 2006, The Preclinical and Clinical Trial of Platelet Substitute—Fibrinoplate, 4^{th} ASIAN PACIFIC CONGRESS ON THROMBOSIS AND HAEMOSTASIS, SUZHOU, CHINA, Sep. 23, 2006.
13) Mao, 2014, Residual Effect of One Dose of Fibrinoplate-S in a Mice Radiation Model, Personal communication, not yet published.
14) Rithidech, 2012, Attenuation of Oxidative Damage and Inflammatory Responses by Apigenin Given to Mice After Irradiation, Mutat Res. 2012 Dec. 12; 749(1-2):29-38. doi: 10.1016/j.mrgentox.2012.08.001. Epub 2012 Aug. 15.
15) Sanchez, 2004, Analysis of Human Peripheral Blood Samples from Fatal and Nonfatal Cases of Ebola (Sudan) Hemorrhagic Fever: Cellular Responses, Virus Load, and Nitric Oxide Levels, J. Virol. October 2004 vol. 78no. 19 10370-10377.
16) Sullivan, 2003, Ebola Virus Pathogenesis: Implications for Vaccines and Therapies, doi: 10.1128/JVI.77.18.9733-9737.2003, J. Virol. September 2003 vol. 77 no. 18 9733-9737.
17) Sung, 2014, Fibrinogen Coasted Nanospheres Prevent Thrombocytopenia-related Bleeding, American Society of Hematologists annual meeting, December, 2014.
18) Winslow, 2013, Oxygen: the Poison is in the Dose, Transfusion. 2013 February; 53(2):424-37, doi: 10.1111/j.1537-2995.2012.03774.x. Epub 2012 Jul. 15.
19) Yang, 1998, Distinct Cellular Interactions of Secreted and Transmembrane Ebola Virus Glycoproteins, Science 1998, February 13:279 (5353):1034-7.
20) Yen, 1995, A Novel Approach to Correcting the Bleeding Associated with Thrombocytopenia, Presented to American Association of Blood Banks: 48^{th} annual meeting, Nov. 11-15, 1995.
21) Yen, 2011, Request for Designation: Fibrinoplate-S is a Biologiical Product and Affirmation from the US FDA, Personal Communication.

What is claimed as being new and desired to be protected by Letters Patent of the United States is as follows:

1. A method of using fibrinogen-coated albumin spheres for treating a patient infected with a hemorrhagic virus, said method comprising the steps of:
a) preparing a protein nanoparticle suspension containing submicron protein spheres;
b) administering a predetermined amount of said protein nanoparticle suspension to the patient infected with a hemorrhagic virus to provide improved hemostatic function of a residual concentration of platelets of the patient resulting in decreasing mortality rate or decreasing morbidity of the patient;
c) protecting an endothelial function of an endothelial cell of a blood vessel of the patient by said protein nanoparticle suspension resulting in improved permeability control across predetermined tissues in the patient; and
d) counteracting an effect of the hemorrhagic virus on a wall of the blood vessel by said protein nanoparticle suspension;
wherein said protein spheres of said protein nanoparticle suspension are bound with fibrinogen molecules in vitro or in vivo.

2. The method according to claim 1, where said step of preparing said protein nanoparticle suspension further comprises the steps of:
adding a predetermined amount of a glutaraldehyde solution to a predetermined amount of an albumin solution to form a mixture;
adding a predetermined amount of a first desolvation solution to said mixture to form a second mixture; and
removing at least a portion of said first desolvation solution from said second mixture.

3. The method according to claim 2, wherein said predetermined amount of said first desolvation solution is configured to result in a concentration of said first desolvation solution insufficient to cause persistent turbidity of said first mixture.

4. The method according to claim 3, wherein said step of preparing said protein nanoparticle suspension further comprises, prior to said step of removing at least a portion of said first desolvation solution from said second mixture, the step of adding a predetermined amount of a second desolvation solution to said second mixture to form a third mixture.

5. The method according to claim 4, wherein said step of removing at least a portion of said first desolvation solution includes a step of removing at least a portion of said second desolvation solution

6. The method according to claim 5, wherein said step of removing at least a portion of said first and second desolvation solutions is conducted by dialysis in a dialysis bag against water.

7. The method according to claim 6 further comprising, after said step of removing at least a portion of said first and second desolvation solutions, the step of adding sorbitol to achieve an osmolarity of said protein nanoparticle suspension compatible with plasma of the patient.

8. The method according to claim 4, wherein said predetermined amount of said second desolvation solution is configured to result in a combined concentration of said first and second desolvation solutions sufficient to cause formation of said protein spheres stable against redissolving and without formation of aggregates.

9. The method according to claim 8, wherein said second desolvation solution is the same as said first desolvation solution.

10. The method according to claim 9, wherein a volume of said second desolvation solution is greater than a volume of said first desolvation solution.

11. The method according to claim 2, wherein said albumin solution is a human serum albumin solution.

12. The method according to claim 1, wherein said fibrinogen molecules are human fibrinogen molecules extracted from sero-negative human plasma.

13. The method according to claim 1, wherein said fibrinogen molecules are bound to said protein spheres in vivo with said fibrinogen molecules being supplied by blood of the patient.

14. The method according to claim 1, wherein said fibrinogen molecules are bound to said protein spheres in vitro with said fibrinogen molecules being supplied by blood.

15. The method according to claim 1, wherein said fibrinogen molecules are bound to said protein spheres in vitro or in vivo using a biological molecule solution containing said fibrinogen molecules and insulin.

16. The method according to claim 1, wherein said step of preparing said protein nanoparticle suspension further comprises the step of adding at least one excipient to said protein nanoparticle suspension to achieve compatibility with osmolarity of blood of the patient.

17. The method according to claim 15, wherein said excipient is selected from the group consisting of sodium caprylate, sorbitol, and a mixture of sodium caprylate and sorbitol.

18. The method according to claim 1, wherein the hemorrhagic virus is Ebola.

19. The method according to claim 1, wherein said step of counteracting an effect of the hemorrhagic virus on a wall of the blood vessel further comprises the step of forming co-aggregates with activated endogenous platelets at sites of injury inside the blood vessel of the patient at an onset of injury.

20. The method according to claim 1, wherein said predetermined amount of said protein nanoparticle suspension administered to the patient is in a range of 8 mg to 16 mg of spheres per kg weight of the patient.
